(19) United States
(12) Patent Application Publication
(10) Pub. No.: US 2026/0250727 A1
(43) Pub. Date: Aug. 27, 2026

(54) METHOD FOR MODIFYING A HIGH-YIELD L-LYSINE-PRODUCING STRAIN ENGINEERING AND APPLICATION THEREOF

(71) Applicant: Jiangnan University, Wuxi (CN)

(72) Inventors: Liming Liu, Wuxi (CN); Haomiao Wang, Wuxi (CN); Jia Liu, Wuxi (CN); Xiaomin Li, Wuxi (CN); Kaifang Liu, Wuxi (CN); Cong Gao, Wuxi (CN); Guipeng Hu, Wuxi (CN)

(21) Appl. No.: 19/669,140

(22) Filed: May 6, 2026

(30) Foreign Application Priority Data
May 27, 2025 (CN) 2025106901067

(51) Int. Cl. C12P 13/08; C12N 15/70

(52) U.S. Cl. CPC C12P 13/08; C12N 15/70

(57) ABSTRACT

The present disclosure relates to the field of biotechnology, and in particular to method for modifying a high-yield L-lysine-producing strain engineering and application thereof. The present disclosure provides a recombinant strain obtained by knocking out the cbrB gene and/or the cbrC gene in the genome of E. coli. Fermentation of the recombinant strain can increase the yield and conversion rate of L-lysine and reduce the mortality rate of the strain during the fermentation process. The present disclosure has significant application value. Through experimental studies, it was found that these two genes have a synergistic effect on E. coli. L-lysine hydrochloride imposes osmotic stress resistance on the strains, and silencing the related gene proteins can alter the membrane lipid composition (increasing a proportion of unsaturated fatty acids), maintain membrane fluidity, improve tolerance to lysine hydrochloride, and reduce the mortality rate in the late stage of fermentation.

### REFERENCE TO SEQUENCE LISTING

The instant application contains a Sequence Listing in XML format as a file named “PC260005A.xml”, created on Apr. 27, 2026, of 64,892 bytes in size, and which is hereby incorporated by reference in its entirety.

### TECHNICAL FIELD

The present disclosure relates to the field of biotechnology, and in particular to method for modifying a high-yield L-lysine-producing strain engineering and application thereof.

### BACKGROUND

Lysine has a chemical name of 2,6-diaminohexanoic acid, and is a basic essential amino acid that cannot be synthesized by the human body and must be obtained from food. Lysine is primarily found in animal-based foods and legumes, while its content in cereal food is relatively low. It is easily degraded during processing, resulting in deficiency. Therefore, it is called the first limiting amino acid.

Lysine holds positive nutritional significance in promoting human growth and development, enhancing immune function, exhibiting antiviral properties, promoting fat oxidation, and alleviating anxiety. It also aids in the absorption of certain nutrients and can synergize with other nutrients to better exert the physiological functions of various nutrients. Lysine can regulate the metabolic balance of the human body. It serves as a structural component for the synthesis of carnitine, which, in turn, promotes the synthesis of fatty acids in cells. The addition of a small amount of lysine to food can stimulate the secretion of pepsin and gastric acid, improve the secretion efficiency of gastric juice, and thus enhance appetite and promote the growth and development of young children. Lysine can also improve calcium absorption and its accumulation in the body, accelerating bone growth. A deficiency in lysine can lead to insufficient secretion of gastric juice, resulting in anorexia and nutritional anemia, which, in turn, can impair central nervous system and cause developmental delay. In the pharmaceutical field, lysine can also be used as an adjuvant drug for diuretics to treat lead poisoning caused by reduced chloride levels in the blood. Furthermore, it can also form salts with acidic drugs (such as salicylic acid) to mitigate adverse reactions. When administered in combination with methionine, it can inhibit severe hypertension. Studies have also shown that supplementation with lysine can accelerate the recovery from herpes infections and inhibit their recurrence. Only L-lysine can be absorbed by organisms. The CAS number for L-lysine is 56-87-1. Due to the widespread application of L-lysine in various fields, its demand is increasing year by year. Therefore, various studies are currently underway to develop highly efficient microbial strains and fermentation process technologies for the production of L-lysine. For example, key genes involved in L-lysine biosynthesis are overexpressed in microbial strains to enhance their activity; or unnecessary genes (such as those responsible for byproducts or toxins that affect cell growth) are deleted from the microbial strains. However, as the demand for L-lysine increases year by year, research remains necessary to effectively enhance L-lysine production capacity.

### SUMMARY

A primary objective of the present disclosure is to increase the yield of L-amino acids. Objectives of the present disclosure are not limited to the subject matter described herein, and other objectives not clearly mentioned herein will be readily apparent to those skilled in the art from the following description.

The present disclosure first provides a method for modifying a producing strain to increase the yield of L-lysine, which is a microorganism capable of in vivo inhibiting or downregulating an expression level and/or activity of inner membrane protein. Specifically, three strains, namely, high-yield L-lysine producing strains Escherichia coli lys-1 (E. coli CCTCC NO: M2019435), lys-2 (E. coli NRRLB-12185), and lys-3 (E. coli GDMCC NO. 1.318), are taken as starting strains, an empty vector fragment is introduced into the gene fragments encoding cbrB and cbrC by using a gene modification system and tool plasmids purchased from Professor Yang Sheng. The E. coli NRRLB-12185 strain is a low-yield L-lysine producing strain and can be obtained from the Agricultural Research Service Culture Collection (NRRL).

The present disclosure provides a high-yield L-lysine producing E. coli FMME-lys2.0. E. coli is derived from the chassis strain FMME-lys (E. coli CCTCC NO: M2019435). By inserting an empty vector fragment, with a sequence shown in SEQ ID NO. 3, into a 32 bp target site within a first 200 bp fragment at the beginning of cbrB of the host strain, cbrB is thus disrupted to reduce the expression of inner membrane protein in E. coli, thereby improving the export efficiency of L-lysine, and forming a strain FMME-lys1.0.

Further, the host strain E. coli is FMME-lys1.0. On this basis, an empty vector fragment is inserted into a 32 bp target site within the first 200 bp fragment at the beginning of cbrC to disrupt cbrC, thereby improving the tolerance of E. coli to lysine hydrochloride and forming a strain FMME-lys2.0.

The present disclosure further provides an engineered E. coli strain, where the engineered E. coli is obtained by silencing a CbrB protein and/or a CbrC protein in the genome of E. coli CCTCC NO: M2019435 used as a chassis strain.

In one embodiment, an amino acid sequence of the CbrB protein is as shown in SEQ ID NO. 1

SEQ ID NO. 1 is as follows:

MSVSRRVIHHGLYFAVLGPLIGVLFLVLYIFFAKEPLVLWVIIHPIFLLLSITTGAIPA
LLTGVMVACLPEKIGSQKRYRCLAGGIGGVVITEIYCAVIVHIKGMASSELFENILSGDSL
VVRIIPALLAGVVMSRIITRLPGLDISCPETDSLS

An amino acid sequence of the CbrC protein is as shown in SEQ ID NO. 2.

SEQ ID NO. 2 is as follows:

MTQNIRPLPQFKYHPKPLETGAFEQDKTVECDCCEQQTSVYYSGPFYCVDEVE
HLCPWCIADGSAAEKFAGSFQDDASIEGVEFEYDEEDEFAGIKNTYPDEMLKELVERTPG
YHGWQQEFWLAHCGDFCVFIGYVGWNDIKDRLDEFANLEEDCENFGIRNSDLAKCLQK
GGHCQGYLFRCLHCGKLRLWGDFS

The present disclosure further provides application of the CbrB protein and/or the CbrC protein in improving L-lysine stress tolerance of E. coli, where the application includes silencing the CbrB protein and/or the CbrC protein in the genome of E. coli.

In one embodiment, the E. coli includes, but is not limited to, E. coli CCTCC NO: M2019435, E. coli NRRLB-12185, and E. coli GDMCC NO. 1.318.

In one embodiment, the amino acid sequence of the CbrB protein is as shown in SEQ ID NO. 1, and the amino acid sequence of the CbrC protein is as shown in SEQ ID NO. 2. In one embodiment, a method of silencing includes replacing a gene fragment of SEQ ID NO. 4 in an upstream sequence of the cbrB gene with a target gene fragment shown in SEQ ID NO. 3; and replacing a gene fragment of SEQ ID NO. 5 in an upstream sequence of the cbrC gene with the target gene fragment shown in SEQ ID NO. 3.

SEQ ID NO. 3:
ttgggtgttgtttgaagtataagttgacatatctgtactaaaagatggcataaattggaagtgtaaggtggcatagtctagtatttaa
ccaaatggttaaatggttgactcaccgggaattcgagataaattgcactgaaatctagaggtcgaaattcacctcgaaagcaagctgataaac
cgatacaattaaaggctccttttggagcctttttttttggagattttcaacgtgaaaaaattattattcgcaattccaagctaattcacctcgaa
agcaagctgataaaccgatacaattaaaggctccttttggagcctttttttttggagattttcaacgtgaaaaaattattattcgcaattccaag
ctctgcctcgcgcgtttcggtgatgacggtgaaaacctctgacacatgcagctcccggagacggtcacagcttgtctgtaagcggatgccgggag
cagacaagcccgtcagggcgcgtcagcgggtgttggcgggtgtcggggcgcagccatgacccagtcacgtagcgatagcggagtgtatg
ggctcgatcccctcggatcgatccccatgtaatgaataaaaagcagtaattaatacatctgtttcatttgaagcgcgaaagctaaagttttcgca
tttatcggatccggctgctaatcgagttaattaaactagtgagctcggtacccggggatcctctagaggtcgaaattcacctcgaaagcaagc
tgataaaccgatacaattaaaggctccttttggagcctttttttttggagattttcaacgtgaaaaaattattattcgcaattccaagctaattc
acctcgaaagcaagctgataaaccgatacaattaaaggctccttttggagcctttttttttggagattttcaacgtgaaaaaattattattcgca
attccaagctctgcctcgcgcgtttcggtgatgacggtgaaaacctctgacacatgcagctcccggagacggtcacagcttgtctgtaagcggat
gccgggagcagacaagcccgtcagggcgcgtcagcgggtgttggcgggtgtcggggcgcagccatgacccagtcacgtagcgatagcgg
agtgtatgggctcgatcccctcggatccaattggtcttaattttccttaattatttttaaagttagactgatttagacttggaaaagcttaatga
ttggagagctaaattgactaatagtattcagtcaagtttaattagttttaagcgatccatgcataactatttctgtacaatgctatattttcacc
aattaataatttttaatcaagcctacattatgaaatatactatacccatttgaactcttctatttgtaccattgtcggtagcaaaaacttatggg
gttttgaacgtcacttaaattgtaagcatttgcgatggaggcgcgtttagagtcaaccttgattctgatatgctccgaatttttggtaagaatat
aagtgtgagagtagctaatgtggatacgcctgagttaagggaaaaatgtgaaaatgaaataactcgttatcatgcaaagtgactaaggttataat
cttccgtttatggcacatagcagccaactaaacttgacagtatttttatgtggttggctttataaaaccagcatttggtaacattatgccaattt
ttacttcaatattatgccaacatacactacactaacggagctgtagcacaataagctcgtttgtacttatgccaacttatacttcaaacaacatt
gg
SEQ ID NO. 4:
ctgtctgccggaaaagatcgggtcacagaaac
SEQ ID NO. 5:
aaagtttgcaggtagttttcaggatgatgcca

The present disclosure further provides a method for increasing a biomass at a fermentation final biomass of high-yield L-lysine producing E. coli, where the method includes: silencing the CbrB protein and/or the CbrC protein in the genome of E. coli, and the high-yield L-lysine producing E. coli is E. coli CCTCC NO: M2019435. In one embodiment, the amino acid sequence of the CbrB protein is as shown in SEQ ID NO. 1, and the amino acid sequence of the CbrC protein is as shown in SEQ ID NO. 2.

In one embodiment, a method of silencing includes replacing a gene fragment of SEQ ID NO. 4 in an upstream sequence of the cbrB gene with a target gene fragment shown in SEQ ID NO. 3; and replacing a gene fragment of SEQ ID NO. 5 in an upstream sequence of the cbrC gene with the target gene fragment shown in SEQ ID NO. 3.

The present disclosure provides a method for constructing the above engineered E. coli strain, where the method includes: silencing the cbrB gene and/or the cbrC gene in the genome using gene editing means.

In one embodiment, the method for silencing the cbrB gene and the cbrC gene may be any one of the following:
(i) knocking out the cbrB gene and the cbrC gene using the gene editing means;

A nucleotide fragment of the cbrB gene (SEQ ID NO. 11):

Ttcttcacactcccttcacttaccccgcttaaattggcgctcaaaggtaagtaaagggagtttgatatgtctgtttcacgtcgggta
atacatcacggactttattttgcagttttaggaccgttaattggtgttctgtttcttgtcctctacatattcttcgcaaaagaaccgctggttct
ttgggtgataatacatccaatttttctcttattgtcgataactacgggagctattcctgcgttcttaaccggtgtaatggttgcgttatcgttgt
ctggctggtggcataggtggcgtcgttatcaccgagatctattgtgcagttattgtacatattaagggcatggcttcctcggagttgtttgaaaa
cattctttctggtgacagtctcgttgtccgcatcattcctgcattgctggcaggtgtggtgatgagcagaatcattacccgtctacccggattgg
atatttcatgtcctgaaacagactctttaagttaagcgggatactttatctttgggctactcaaaagcagacaggatgtttct

Specifically, an underlined sequence indicates the cbrB nucleotide sequence to be knocked out.

A nucleotide fragment of the cbrC gene (SEQ ID NO. 12):

gcgggatactttatctttgggctactcaaaagcagacaggatgtttctatgactcaaaatatcaggccgttaccccaattcaaatat
catcccaagccactggaaacaggcgcatttgaacaggataaaaccgtagagtgcgattgctgtgaacaacagacgtcagtttattactcggg
tcccttttattgcgttgatgaagttgaacatctctgtccgtggtgtattgcggacggttctgctgctgagcatagaaggtgttgaatttgagtat
gatgaagaggacgaatttgccggtattaagaacacatatcctgatgaaatgctgaaagagttggttgaacgcacgccaggttatcatggatggc
agcaggaattctggctcgcgcattgtggcgatttctgtgtttttatcggctatgtgggctggaatgatataaaagatcgcctcgatgaatttgcc
aaccttgaagaagattgtgagaatttcggtattagaaattctgatctagctaaatgcctgcaaaagggtggtcattgtcagggttatctcttccg
ctgtctccactgcggcaagctgagactgtggggtgatttttcgtagttatttaaataatgagaacaggccg

Specifically, an underlined sequence indicates the cbrB nucleotide sequence to be knocked out.
(ii) replacing the gene fragment of SEQ ID NO. 4 in the upstream sequence of the cbrB gene with the target gene fragment shown in SEQ ID NO. 3; and
replacing the gene fragment of SEQ ID NO. 5 in the upstream sequence of the cbrC gene with the target gene fragment shown in SEQ ID NO. 3.

In one embodiment, the method includes:
(i) construction of a pDONOR-empty vector plasmid: performing PCR with primers using the genome of E. coli as a template, using a stop codon as a target gene fragment, and using 20 bp at each end of the target gene fragment as homologous arms to construct a target gene fragment having the nucleotide sequence as shown in SEQ ID NO. 3; performing inverse PCR with the primers to form a vector backbone of the pDONOR plasmid, and performing homologous recombination of the two fragments using a homologous recombinase to form the pDONOR-empty vector plasmid;
(ii) construction of pQCascade-cbrB and pQCascade-cbrC plasmids: identifying a 32 bp target sequence within 200 bp upstream of the cbrB and cbrC fragments, respectively, with the target sequences as shown in SEQ ID NO. 4 and SEQ ID NO. 5; replacing the 32 bp sequence shown in SEQ ID NO. 10 in the original pQCascade plasmid with the target sequences by inverse PCR using primers, respectively to construct the pQCascade-cbrB and pQCascade-cbrC plasmids;
(iii) construction of an engineered E. coli strain with the cbrB gene silenced: electrotransforming the pDONOR-empty vector plasmid and pQCascade-cbrB into the chassis strain E. coli CCTCC NO: M2019435, respectively, inducing with L-rhamnose, successfully inserting the empty vector plasmid into the region 200 bp upstream of the cbrB gene, and curing the plasmids to obtain an engineered E. coli strain with the cbrB gene silenced;
(iv) construction of an engineered E. coli strain with the cbrC gene silenced: electrotransforming the pDONOR-empty vector plasmid and pQCascade-cbrC into the chassis strain E. coli CCTCC NO: M2019435, respectively, inducing with L-rhamnose, successfully inserting the empty vector plasmid into the region 200 bp upstream of the cbrC gene, and curing the plasmids to obtain an engineered E. coli strain with the cbrC gene silenced; and
(vi) construction of an engineered E. coli strain with both the cbrB gene and the cbrC gene silenced:
electrotransforming the pDONOR-empty vector plasmid and pQCascade-cbrC respectively into the engineered E. coli strain with the cbrB gene silenced constructed in the step (iii), inducing with L-rhamnose, successfully inserting the empty vector plasmid into the region 200 bp upstream of the cbrC gene, and curing the plasmids to obtain an engineered E. coli strain with the cbrB gene and the cbrC gene silenced.

A nucleotide sequence fragment after silencing the cbrB gene is as follows (SEQ ID NO. 13):

atgtctgtttcacgtcgggtaatacatcacggactttattttgcagttttaggaccgttaattggtgttctgtttcttgtcctctacatatt
cttcgcaaaagaaccgctggttctttgggtgataatacatccaatttttctcttattgtcgataactacgggagctattcctgcgttgttaaccg
gtgtaatggttgcttgggtgttgtttgaagtataagttgacatatctgtactaaaagatggcataaattggaagtgtaaggtggcatagtctagt
atttaaccaaatggttaaatggttgactcaccgggaattcgagataaattgcactgaaatctagaggtcgaaattcacctcgaaagcaagctgat
aaaccgatacaattaaaggctccttttggagccttttttttggagattttcaacgtgaaaaaattattattcgcaattccaagctaattcacctc
gaaagcaagctgataaaccgatacaattaaaggctccttttggagccttttttttggagattttcaacgtgaaaaaattattattcgcaattcca
agctctgcctcgcgcgtttcggtgatgacggtgaaaacctctgacacatgcagctcccggagacggtcagcttgtctgtaagcggatgccgggag
cagacaagcccgtcagggcgcgtcagcgggtttggcgggtgtcggggcgcagccatgacccagtcacgtagcgatagcggagtgtatgggc
tcgatcccctcggatcgatccccatgtaatgaataaaagcagtaattaatacatctgtttcatttgaagcgcgaaagctaaagttttcgcattta
tcggatccggctgctaatcgagttaattaaactagtgagctcggtacccggggatcctctagaggtcgaaattcacctcgaaagcaagctgata
aaccgatacaattaaaggctccttttggagcctttttttttggagattttcaacgtgaaaaaattattattcgcaattccaagctaattcacctc
gaaagcaagctgataaaccgatacaattaaaggctccttttggagcctttttttttggagattttcaacgtgaaaaaattattattcgcaattcc
aagctctgcctcgcgcgtttcggtgatgacggtgaaaacctctgacacatgcagctcccggagacggtcacagcttgtctgtaagcggatgccgg
gagcagacaagcccgtcagggcgcgtcagcgggtcttggcgggtgtcggggcgcagccatgacccagtcacgtagcgatagcggagtgta
tgggctcgatcccctcggatccaattggtcttaattttccttaattatttttaaagttagactgatttagacttggaaaagcttaatgattggag
agctaaattgactaatagtattcagtcaagtttaattagttttaagcgatccatgcataactatttctgtacaatgctatattttcaccaattaa
taatttttaatcaagcctacattatgaaatatactatacccatttgaactcttctatttgtaccattgtcggtagcaaaaacttatggggttttg
aacgtcacttaaattgtaagcatttgcgatggaggcgcgtttagagtcaaccttgattctgatatgctccgaatttttggtaagaatataagtgt
gagagtagctaatgtggatacgcctgagttaagggaaaaatgtgaaaatgaaataactcgttatcatgcaaagtgactaaggttataatcttccg
tttatggcacatagcagccaactaaacttgacagtatttttatgtggttggctttataaaaccagcatttggtaacattatgccaatttttactt
caatattatgccaacatacactacactaacggagctgtagcacaataagctcgtttgtacttatgccaacttatacttcaaacaacattgggtta
tcgttgtctggctggtggcataggtggcgtcgttatcaccgagatctattgtgcagttattgtacatattaagggcatggcttcctcggagttgt
ttgaaaacattctttctggtgacagtctcgttgtccgcatcattcctgcattgctggcaggtgtggtgatgagcagaatcattacccgtctaccc
ggattggatatttcatgtcctgaaacagactctttaagttaa

(Note: the underlined sequence indicates the inserted empty vector fragment, and the non-underlined sequence indicates the gene fragment of cbrB.)

A nucleotide sequence fragment after silencing the cbrC gene is as follows (SEQ ID NO. 14):

atgactcaaaatatcaggccgttaccccaattcaaatatcatcccaagccactggaaacaggcgcatttgaacaggataaaacc
gtagagtgcgattgctgtgaacaacagacgtcagtttattactcgggtcccttttattgcgttgatgaagttgaacatctctgtccgtggtgtat
tgcggacggttctgctgctgattgggtgttgtttgaagtataagttgacatatctgtactaaaagatggcataasttggaagtgtaaggtggcat
agtctagtatttaaccaaatggttaaatggttgactcaccgggaattcgagataaattgcactgaaatctagaggtcgaaattcacctcgaaagc
aagctgataaaccgatacaattaaaggctccttttggagccttttttttggagattttcaacgtgaaaaaattattattcgcaattccaagctaa
ttcacctcgaaagcaagctgataaaccgatacaattaaaggctccttttggagccttttttttggagattttcaacgtgaaaaaattattattcg
caattccaagctctgcctcgcgcgtttcggtgatgacggtgaaaacctctgacacatgcagctcccggagacggtcagcttgtctgtaagcggat
gccgggagcagacaagcccgtcagggcgcgtcagcgggtttggcgggtgtcggggcgcagccatgacccagtcacgtagcgatagcgga
gtgtatgggctcgatcccctcggatcgatccccatgtaatgaataaaagcagtaattaatacatctgtttcatttgaagcgcgaaagctaaagtt
ttcgcatttatcggatccggctgctaatcgagttaattaaactagtgagctcggtacccggggatcctctagaggtcgaaattcacctcgaaag
caagctgataaaccgatacaattaaaggctccttttggagcctttttttttggagattttcaacgtgaaaaaattattattcgcaattccaagct
aattcacctcgaaagcaagctgataaaccgatacaattaaaggctccttttggagcctttttttttggagattttcaacgtgaaaaaattattat
tcgcaattccaagctctgcctcgcgcgtttcggtgatgacggtgaaaacctctgacacatgcagctcccggagacggtcacagcttgtctgtaag
cggatgccgggagcagacaagcccgtcagggcgcgtcagcgggtgttggcgggtgtcggggcgcagccatgacccagtcacgtagcgata
atgattggagagctaaattgactaatagtattcagtcaagtttaattagttttaagcgatccatgcataactatttctgtacaatgctatatttt
caccaattaataatttttaatcaagcctacattatgaaatatactatacccatttgaactcttctatttgtaccattgtcggtagcaaaaactta
tggggttttgaacgtcacttaaattgtaagcatttgcgatggaggcgcgtttagagtcaaccttgattctgatatgctccgaatttttggtaaga
atataagtgtgagagtagctaatgtggatacgcctgagttaagggaaaaatgtgaaaatgaaataactcgttatcatgcaaagtgactaaggtta
taatcttccgtttatggcacatagcagccaactaaacttgacagtatttttatgtggttggctttataaaaccagcatttggtaacattatgcca
atttttacttcaatattatgccaacatacactacactaacggagctgtagcacaataagctcgtttgtacttatgccaacttatacttcaaacaa
cattgggcatagaaggtgttgaatttgagtatgatgaagaggacgaatttgccggtattaagaacacatatcctgatgaaatgctgaaagagttg
gttgaacgcacgccaggttatcatggatggcagcaggaattctggctcgcgcattgtggcgatttctgtgtttttatcggctatgtgggctggaa
tgatataaaagatcgcctcgatgaatttgccaaccttgaagaagattgtgagaatttcggtattagaaattctgatctagctaaatgcctgcaaa
agggtggtcatgtcagggttatctcttccgctgtctccactgcggcaagctgagactgtggggtgatttttcgtag

(Note: the underlined sequence indicates the inserted empty vector fragment, and the non-underlined sequence indicates the gene fragment of cbrC.)

The present disclosure further provides a method for fermentative production of L-lysine, where the method includes fermentative production of L-lysine using the engineered E. coli strain in which the cbrB gene and/or the cbrC gene are/is silenced.

In one embodiment, a fermentation temperature is 30-37° C., and a fermentation duration is 30-42 hours; in one embodiment, the method includes: inoculating a seed culture of the engineered E. coli strain into a fermentation medium containing glucose, followed by fermentation; further, the seed culture is added at a volume ratio of 15-20% (v/v).

In one embodiment, a preparation method of the seed culture includes: streaking the engineered E. coli strain onto an agar slant medium, culturing at a temperature of 33-37° C. for 12-16 hours, inoculating into a primary seed medium at an inoculum size of 5-15% (v/v), culturing at a temperature of 33-37° C. until OD₅₆₂ reaches 4.0-5.0 to obtain a primary seed culture, inoculating the obtained primary seed culture into a secondary seed medium at an inoculum size of 5-15% (v/v), and culturing at a temperature of 33-37° C. until OD₅₆₂ reaches 15.0-18.0.

The fermentative production of L-lysine using the engineered E. coli strain in which the cbrB gene and/or the cbrC gene are/is silenced in the method include: streaking the engineered E. coli strain in which the cbrB gene and/or the cbrC gene are/is silenced onto an agar slant medium and culturing at a temperature of 33-37° C. for 12-16 hours; inoculating into a primary seed medium at an inoculum size of 5-15% (v/v), shaking culture at 33-37° C. for 5-8 hours until reaching a mid-log phase with an OD₅₆₂ of 4-5; inoculating into a secondary seed medium at an inoculum size of 5-15% (v/v), shaking culture for 5-8 hours until reaching a mid-log phase with an OD₅₆₂ of 15-18; inoculating into a fermentation medium at an inoculum size of 15-20% (v/v), controlling an aeration rate at 3-7 vvm, controlling a temperature at 33-37° C., and continuing stirring at 600-800 rpm, and terminating the fermentation immediately after glucose is depleted.

The agar slant medium includes: yeast extract (Oxoid) 5 g/L, tryptone (Oxoid) 10 g/L, sodium chloride 5 g/L, agar powder 20 g/L, sodium pyruvate 0.5 g/L; and sterilized at 121° C. for 15 minutes.

Components of the primary seed medium include: sucrose 3.0 g/L, yeast extract (Oxoid) 5 g/L, tryptone (Oxoid) 8 g/L, (NH4)₂SO₄ 4.5 g/L, K₂HPO₄ 4 g/L, MgSO4·7H₂O 0.5 g/L, FeSO₄·7H₂O 22 mg/L, MnSO₄·H₂O 13 mg/L, sodium glutamate 5 g/L, L-threonine 0.3 g/L, L-methionine 0.3 g/L, sodium pyruvate 0.55 g/L, and biotin 5 mg/L.

Components of the secondary seed medium include: glucose 50 g/L, (NH₄)₂SO₄ 14.4 g/L, KH₂PO₄ 1.44 g/L, MgSO₄·7H₂O 1.2 g/L, corn steep liquor powder 5.92 g/L, hair hydrolysate 3.12 g/L, FeSO₄·7H₂O 24.9 mg/L, MnSO₄·H₂O 15.2 mg/L, ZnSO₄·7H₂O 102.4 mg/L, CuSO₄·5H₂O 80.6 mg/L, L-threonine 368 mg/L, L-methionine 368 mg/L, betaine hydrochloride 1.6 g/L, PABA 2 mg/L, VB1 1 mg/L, nicotinamide 1 mg/L, biotin 5 mg/L, light calcium carbonate (1,250 mesh) 10 g/L.

Components of the fermentation medium include: glucose 30 g/L, 85% H₃PO₄ 0.2 mL/L, potassium chloride 0.5 g/L, beet molasses 18 mL/L, betaine hydrochloride 1.5 g/L, MgSO₄·7H₂O 3.29 g/L, FeSO₄·7H₂O 49.4 mg/L, MnSO₄·H₂O 35.8 mg/L, ZnSO₄·7H₂O 152.9 mg/L, CuSO₄·5H₂O 120 mg/L, L-threonine 300 mg/L, corn steep liquor powder 7.4 g/L, defoamer 2 g/L, VB1 60 mg/L, nicotinamide 10 mg/L, biotin 0.6 mg/L.

A fourth objective of the present disclosure is to provide a microbial preparation containing the engineered E. coli strain in which the cbrB gene and/or the cbrC gene are/is silenced.

The present disclosure further provides application of the microbial preparation containing the engineered E. coli strain in which the cbrB gene and/or the cbrC gene are/is silenced in food or pharmaceuticals.

Optionally, the application includes producing L-lysine using the microbial preparation containing the engineered E. coli strain in which the cbrB gene and/or the cbrC gene are/is silenced.

The present disclosure further provides the application of the above method for constructing the engineered E. coli strain or the above engineered E. coli strain in any one of the following:
(a) application in the preparation of L-lysine or food, pharmaceuticals, chemicals, or feed containing L-lysine;
(b) application in improving L-lysine stress tolerance of E. coli;
(c) application in increasing a biomass at the fermentation final biomass of high-yield L-lysine producing E. coli or reducing a mortality rate during the fermentation process of high-yield L-lysine producing E. coli; and
(d) application in regulating the yield of L-lysine in E. coli.

In one embodiment, the pharmaceuticals further include a pharmaceutically acceptable excipient; and the pharmaceutically acceptable excipient refers to conventional pharmaceutical carriers in the pharmaceutical field.

In one embodiment, the excipient includes one or more of the following: binders such as cellulose derivatives, alginates, gelatin and polyvinylpyrrolidone; diluents such as starch, pregelatinized starch, dextrin, sucrose, lactose, and mannitol; fillers such as starch and sucrose; wetting agents such as glycerol; disintegrants such as sodium carboxymethyl starch, crosslinked polyvinylpyrrolidone, and dry starch; absorption enhancers such as quaternary ammonium compounds; surfactants such as polysorbates, sorbitan fatty acid esters, and glycerol fatty acid esters; colorants such as titanium dioxide, sunset yellow, methylene blue, and medicinal red iron oxide; lubricants such as hydrogenated vegetable oils, talc, and polyethylene glycol; coating materials such as acrylic resins, hydroxypropyl methylcellulose, polyvinylpyrrolidone, and cellulose acetate esters; additionally, other adjuvants such as flavoring agents and sweeteners may also be added to the composition.

In one embodiment, a dosage form of the pharmaceuticals includes, but is not limited to, oral dosage forms, injectable dosage forms, and inhalation dosage forms.

In one embodiment, the oral dosage forms include, but are not limited to, tablets, capsules, granules, oral liquids, and oral suspensions.

In one embodiment, the injectable dosage forms include, but are not limited to, injectable solutions and injectable powders.

In one embodiment, the inhalation dosage forms include, but are not limited to, aerosols and dry powder inhalers.

The present disclosure further provides application of the CbrB protein having the amino acid sequence as shown in SEQ ID NO. 1 and/or the CbrC protein having the amino acid sequence as shown in SEQ ID NO. 2 in improving the yield of L-lysine produced by E. coli, where the application includes silencing the CbrB protein and/or the CbrC protein in the genome of E. coli.

In one embodiment, the E. coli includes, but is not limited to, E. coli CCTCC NO: M2019435, E. coli NRRLB-12185, and E. coli GDMCC NO. 1.318.

### Beneficial Effects

E. coli is the most commonly used cell factory with a well-defined genetic background. In order to further increase the yield of L-lysine, the beneficial effects of silencing the CbrB protein and/or the CbrC protein in producing strains are mainly reflected in the following two aspects:

First, three different L-lysine producing strains were subjected to silencing of the CbrB protein and/or the CbrC protein, and three parallel experiments were performed for each strain. The conversion rate and yield of L-lysine were both significantly improved. Specifically, the lysine shake flask yield of the lys-1 strain increased by about 18.37%, that of the lys-2 strain increased by about 38.9%, and that of the lys-3 strain increased by about 21.32%, as shown in FIG. 1.

In addition, these two genes have a synergistic effect. L-lysine hydrochloride imposes osmotic stress resistance on the strains, and silencing the related gene proteins can alter the membrane lipid composition (increasing a proportion of unsaturated fatty acids), maintain membrane fluidity, improve tolerance to lysine hydrochloride, and reduce the mortality rate in the late stage of fermentation.

### BRIEF DESCRIPTION OF THE DRAWINGS

FIG. 1 is a yield comparison chart.

FIG. 2 is a diagram of mortality rate during fermentation.

FIG. 3 is an osmotic pressure comparison chart of final fermentation samples.

FIG. 4 is a schematic diagram of site-specific transposition mediated by a CRISPR-associated transposase.

### DETAILED DESCRIPTIONS OF THE EMBODIMENTS

In the following examples, conventional experimental methods are used, and all materials are commercially available. The original plasmids of the tools pQCascade (abbreviated as PQ), pDonor-GFP (abbreviated as PD), and Pcut plasmid was purchased from addgene or Molecular Cloud (https://www.addgene.org), with Purchase No. as follows: Addgene (No.: 140622-140632) and Molecular Cloud (No.: 101225-101235) PDONOR-GFP (175578) and PQ-IS186 (175581).

pDonor-GFP is a commercially available plasmid. The pQ plasmid carries a kanamycin resistance gene, and the PD plasmid carries a chloramphenicol (Cm) resistance gene. The strains harboring the relevant plasmids are based on the JM109 strain.

After extensive experimentation, the inventors of the present disclosure used the L-lysine producing strain FMME-lys (E. coli FMME-lys) as a starting strain for modification to obtain an engineered strain FMME-lys1.0, a genotype of the engineered strain is FMME-lys-AcbrB. FMME-lys1.0 was then used as a chassis strain, an iterative modification was performed to obtain an FMME-lys2.0 strain, a genotype of FMME-lys2.0 is FMME-lys-AcbrBAcbrC.

The E. coli FMME-lys is E. coli CCTCC NO: M2019435, which is disclosed in the Chinese invention patent with Publication NO. CN 110964670B.

### Detection Methods Involved in the Following Examples are as Follows:

### Amino Acid Detection

Detection was performed using a conventional ninhydrin colorimetric method. For further details, please refer to Study on Conditions for Quantitative Detection of Lysine by Ninhydrin Colorimetry, China FoodAdditives, By Liu Feifei.

### Glucose Determination Method:

Analysis was conducted using an SBA-40 biosensor analyzer. For further details, please refer to Study on Factors Influencing the Determination of Glucose and L-lactic Acid by Biosensor Analyzer, Asia-Pacific Traditional Medicine, by Li Xianmin.

### Calculation of Glucose Yield:

Glucose yield (g/g)=Maximum L-lysine production (g/L)/Total glucose added.

### Testing of Strain Mortality Rate:

The instrument used for testing is a flow cytometer (model: Attune CytPix).

### Sample Processing:

Cell collection: Adherent cells: cells were digested with trypsin without EDTA (EDTA may induce apoptosis). The digestion time should not be too long, and gentle pipetting should be performed to avoid mechanical damage. Suspension cells: the cell suspension was directly collected, avoiding vigorous shaking or high-speed centrifugation (Recommended centrifugation speed: 300-400×g for 5 minutes).

Cell washing: The cells were washed 1-2 times with pre-cooled PBS (containing 1% BSA or fetal bovine serum) to remove dead cell debris and serum interference in the medium. A supernatant after centrifugation was discarded and the cells were gently resuspended.

Staining (selection of appropriate dyes): Nucleic acid dye method (suitable for distinguishing dead/live cells): Propidium iodide (PI): final concentration of 1-5 μg/mL, incubated in a dark for 15-30 minutes (4° C. or room temperature). 7-Aminoactinomycin D (7-AAD): final concentration 5-20 μg/mL, incubated in a dark for 20 minutes (room temperature). 4′,6-diamidino-2-phenylindole (DAPI): final concentration 0.1-1 μg/mL (requires membrane permeabilization, used only for fixed cells). Membrane integrity assays (such as trypan blue, but not commonly used in flow cytometry and requires microscopy assistance).

Principle of strain mortality assay: PI labeled dead cells with damaged membranes (late apoptosis/necrosis). Annexin V labeled early apoptotic cells (phospholipid externalization with preserved membrane integrity).

### Calculation of Mortality Rate (%):

Mortality rate (%)=Number of dead cells/(Number of dead cells+Number of live cells)×100%

Note: Depending on the experimental objectives, only PI-positive cells (necrotic cells) may be counted, or Annexin V may be used in combination to distinguish apoptosis stages.

### Calculation Method of Conversion Rate (%):

Definition: Conversion rate (%)=Mass of product (g)/Mass of consumed substrate (g)×100%

During a fermentation process, the substrate involved is glucose and the product is L-lysine; therefore, a calculation formula for the conversion rate is as follows:

### Calculation Formula for the Conversion Rate:

$$Conversion rate ( \% ) = \frac{Lysine yield ( g )}{Mass of glucose consumed ( g )} \times 100 \%$$

### The Media Involved in the Following Examples are as Follows:

### Components of an Agar Slant Medium Include:

Yeast extract (Oxoid) 5 g/L, tryptone (Oxoid) 10 g/L, sodium chloride 5 g/L, agar powder 20 g/L, sodium pyruvate 0.5 g/L; sterilized at 121° C. for 15 minutes.

### Components of a Primary Seed Medium Include:

Sucrose 3.0 g/L, yeast extract (Oxoid) 5 g/L, tryptone (Oxoid) 8 g/L, (NH₄)₂SO₄ 4.5 g/L, K₂HPO₄ 4 g/L, MgSO₄·7H₂O 0.5 g/L, FeSO₄·7H₂O 22 mg/L, MnSO₄·H₂O 13 mg/L, monosodium glutamate 5 g/L, L-threonine 0.3 g/L, L-methionine 0.3 g/L, sodium pyruvate 0.55 g/L, biotin 5 mg/L; sterilized at 121° C. for 15 minutes.

### Components of a Secondary Seed Medium Include:

Glucose 50 g/L, (NH₄)₂SO₄ 14.4 g/L, KH₂PO₄ 1.44 g/L, MgSO₄·7H₂O 1.2 g/L, corn steep liquor powder 5.92 g/L, hair hydrolysate 3.12 g/L, FeSO₄·7H₂O 24.9 mg/L, MnSO₄·H₂O 15.2 mg/L, ZnSO₄·7H₂O 102.4 mg/L, CuSO₄·5H₂O 80.6 mg/L, L-threonine 368 mg/L, L-methionine 368 mg/L, betaine hydrochloride 1.6 g/L, PABA2 mg/L, VB1 1 mg/L, nicotinamide 1 mg/L, biotin 5 mg/L, light calcium carbonate (1,250 mesh) 10 g/L; adjusted a pH to 7.6-7.7 with ammonia water, and sterilized at 121° C. for 15 minutes.

### Components of a Fermentation Medium Include:

Glucose 30 g/L, 85% H₃PO₄ 0.2 mL/L, potassium chloride 0.5 g/L, beet molasses 18 mL/L, betaine hydrochloride 1.5 g/L, MgSO₄·7H₂O 3.29 g/L, FeSO₄·7H₂O 49.4 mg/L, MnSO₄·H₂O 35.8 mg/L, ZnSO₄·7H₂O 152.9 mg/L, CuSO₄·5H₂O 120 mg/L, L-threonine 300 mg/L, corn steep liquor powder 7.4 g/L, defoamer 2 g/L, VB1 60 mg/L, nicotinamide 10 mg/L, biotin 0.6 mg/L; adjusted a pH to 6.6-6.7 with ammonia water.

The primers involved in the following examples are shown in Table 1:

TABLE 1
Primers

| S/N | Base sequence |
|---|---|
| P1 | ggttgactcaccgggaattcGAGATAAATTGCACTGAAATCTAGAGG (SEQ ID |
| | NO. 15) |
| P2 | ctcgattagcagccggatccGATAAATGCGAAAACTTTAGCTTTCGCGCTTC |
| | (SEQ ID NO. 16) |
| P3 | ggatccggctgctaatcgagttaattaa (SEQ ID NO. 17) |
| P4 | gaattcccggtgagtcaacc (SEQ ID NO. 18) |
| P5 | ctgtctgccggaaaagatcgggtcacagaaacgtgaactgccgagtaggcagctgaagttggatccgaattcga |
| | gctcggg (SEQ ID NO. 19) |
| P6 | agaacttatcggtggtggtgtcacg (SEQ ID NO. 20) |
| P7 | gtttctgtgacccgatcttttccggcagacagatttccagctgcctactcggcagttcacccatggtatatctcct |
| | tttaaagt (SEQ ID NO. 21) |
| P8 | cgtgacaccaccaccgataagttct (SEQ ID NO. 22) |
| P9 | aaagtttgcaggtagttttcaggatgatgccagtgaactgccgagtaggcagctgaagttggatccgaattcgagc |
| | tcggg (SEQ ID NO. 23) |
| P10 | agaacttatcggtggtggtgtcacg (SEQ ID NO. 24) |
| P11 | tggcatcatcctgaaaactacctgcaaactttatttccagctgcctactcggcagttcacccatggtatatctcct |
| | tttaaagt (SEQ ID NO. 25) |
| P12 | cgtgacaccaccaccgataagttct (SEQ ID NO. 26) |
| PD-YZ-s | cggaagctttaatgcgggtcct (SEQ ID NO. 27) |
| PD-YZ-x | gatgcgtccggcgtagaggatccacc (SEQ ID NO. 28) |
| PQ-YZ | GGCGAGTTTACGGGTTGTTA (SEQ ID NO. 29) |
| PJY-YZ-S-1 | gcgctcaaaggtaagtaaagggagtttgat (SEQ ID NO. 30) |
| PJY-YZ-X-2 | tgagtagcccaaagataaagtatcccgc (SEQ ID NO. 31) |
| PJY-YZ-S-3 | ctactcaaaagcagacaggatgtttct (SEQ ID NO. 32) |
| PJY-YZ-S-4 | cggcctgttctcattatttaaataa (SEQ ID NO. 33) |

### Example 1: Construction of a Strain with Silenced cbrB Gene in the Genome of an L-Lysine Producing Strain

Three L-lysine producing strains (lys-1: E. coli CCTCC NO: M2019435; lys-2: E. coli NRRLB-12185; lys-3: E. coli GDMCC NO. 1.318) were used as chassis strains for independent genetic modifications. Using an MUCICAT gene editing method, a target gene (terminator) with a nucleotide sequence as shown in SEQ ID NO. 3 was used to replace a 32 bp target sequence (ctgtctgccggaaaagatcgggtcacagaaac, SEQ ID NO. 4) located upstream of a cbrB gene in the L-lysine producing strains. The specific steps are as follows:

### 1. Construction of an Expression Vector Using the MUCICAT Gene Editing Method

(1) acquisition of different fragments:

Using a model E. coli strain MG1655 as a template, PCR amplification was performed with primers P1 and P2, and the amplified product was recovered to obtain a fragment of the target gene (terminator) with a nucleotide sequence as shown in SEQ ID NO. 3.

SEQ ID NO. 3:
ttgggtgttgtttgaagtataagttgacatatctgtactaaaagatggcataaattggaagtgtaaggtggcatagtctagtatttaa
ccaaatggttaaatggttgactcaccgggaattcgagataaattgcactgaaatctagaggtcgaaattcacctcgaaagcaagctgataaac
cgatacaattaaaggctccttttggagcctttttttttggagattttcaacgtgaaaaaattattattcgcaattccaagctaattcacctcga
aagcaagctgataaaccgatacaattaaaggctccttttggagcctttttttttggagattttcaacgtgaaaaaattattattcgcaattcca
agctctgcctcgcgcgtttcggtgatgacggtgaaaacctctgacacatgcagctcccggagacggtcacagcttgtctgtaagcggatgccgg
gagcagacaagcccgtcagggcgcgtcagcgggtgttggcgggtgtcggggcgcagccatgacccagtcacgtagcgatagcggagtgtatg
ggctcgatcccctcggatcgatccccatgtaatgaataaaaagcagtaattaatacatctgtttcatttgaagcgcgaaagctaaagttttcgc
atttatcggatccggctgctaatcgagttaattaaactagtgagctcggtacccggggatcctctagaggtcgaaattcacctcgaaagcaagc
tgataaaccgatacaattaaaggctccttttggagcctttttttttggagattttcaacgtgaaaaaattattattcgcaattccaagctaatt
cacctcgaaagcaagctgataaaccgatacaattaaaggctccttttggagcctttttttttggagattttcaacgtgaaaaaattattattcg
caattccaagctctgcctcgcgcgtttcggtgatgacggtgaaaacctctgacacatgcagctcccggagacggtcacagcttgtctgtaagcg
gatgccgggagcagacaagcccgtcagggcgcgtcagcgggtgttggcgggtgtcggggcgcagccatgacccagtcacgtagcgatagcgg
agtgtatgggctcgatcccctcggatccaattggtcttaattttccttaattatttttaaagttagactgatttagacttggaaaagcttaatg
attggagagctaaattgactaatagtattcagtcaagtttaattagttttaagcgatccatgcataactatttctgtacaatgctatattttca
ccaattaataatttttaatcaagcctacattatgaaatatactatacccatttgaactcttctatttgtaccattgtcggtagcaaaaacttat
ggggttttgaacgtcacttaaattgtaagcatttgcgatggaggcgcgtttagagtcaaccttgattctgatatgctccgaatttttggtaaga
atataagtgtgagagtagctaatgtggatacgcctgagttaagggaaaaatgtgaaaatgaaataactcgttatcatgcaaagtgactaaggtt
ataatcttccgtttatggcacatagcagccaactaaacttgacagtatttttatgtggttggctttataaaaccagcatttggtaacattatgc
caatttttacttcaatattatgccaacatacactacactaacggagctgtagcacaataagctcgtttgtacttatgccaacttatacttcaaa
caacattgg

Using an original PD-GFP plasmid as a template, reverse PCR amplification of the plasmid was performed with primers P3 and P4 to obtain a vector fragment of the pDONOR plasmid: PD-vector.

Using the original PQ plasmid as a template, PCR amplification was performed with primers P5, P6, P7, and P8 to obtain a PQ-CbrB-1 fragment (SEQ ID NO. 6) and a PQ-CbrB-2 fragment (SEQ ID NO. 7).

SEQ ID NO. 6:
ctgtctgccggaaaagatcgggtcacagaaacgtgaactgccgagtaggcagctgaagttggatccgaattcgagctcggga
gaacttatcggtggtggtgtcacgctgtctgccggaaaagatcgggtcacagaaacgtgaactgccgagtaggcagctgaagttggatccg
aattcgagctcgggaagaaggagatatacatatgcactttctggtgcagaccaagagctacccggacgaggcgctggaaagctatctgctg
cgtctggcgcgtgataacagctacaacggttatagcgagctggcggacatcctgtggcagtggctggcggaacaagataacgagctgga
aggtgcgctgccgctggcgctgagcaaggtggacgtttaccacgcgcgtcaggcgagcagcttccgtatccgtgcgctgaaactggtggc
gcaactggcggacgttaacgcgggtgatattctggcgctggcgtggcgtcgtagcaacttcaagtttggcaacctggcggcggtgagccgt
aacgagctggcgatcccgctggaactgctgcgtaccgataacatcccggtttgcattaaatgcctgagcgagagcagccacattccgtttta
ctggcacctgaagccgtataaagcgtgccacaagcacaaaagccagctgatcacccgttgcaaggagtgctacgacctgattgattatcgt
gcgagcgaggcgtttctggaatgcgtttgcggttgcaaaatcaccaacagcgaacaactgaacgacgcggatttcaagatcgcgattgcgc
tggcgagcagcaacagccagaaaatcgtgggcctgattagctggttcgcgaaggtgaaacaactggacgttagcgacgcggatttcaact
gcgcgtttgttgattacttcaacacctggccggagagcctgaccaccgaactggacctgctgaccaacaacgcgcgtctgaagcagctgaa
cccgtttaacaagaccaaattcagcagcgtgtacggtgacctgatccgtgatggccaaattgcggcgaccagcaaccgtaagaacaaagtt
atcgacgagatcattagctattttgtggaactggttgatagcaacccgaaggcgaaacacccgaacattggtgacctgctgctgtgcaccttc
gatgcggcggtgctgctgaacaccaccaccgagcaggtttaccgtctgcaccaagaagcgtttctgaactgcgcgtatagccagaagaaa
cacgaacaactgcgtgcggatagccacgtgttctatctgcgtcaggttatcgagctgcagcaagcgtttgcggcggaaaaaccgctgacca
agaaacaattcattgcgccgtggtaacttatgaacctgcaggatgcgctggcgattgagccgctgaaggaaaaaaccaccgcgctgcgtaa
gctgttcgtgccgtacaccagccacgttgaggtggatggttttgaggaactggcgctgaccgtgctgatcaacctggtttataagcgtagcga
aattgacgatctgaccagcgcgcgtaccgcgaaaagcgtgctgcgtgacgaggttctgctgagcaagtgcatcaacgaagtgaaatggttc
cacacccacaacctgaagtacccggacatccgtgttagccaccaacgtctgattagcgaggtggttagcgaagatatcgcgggtatttgca
gccgtagcctgccgctgagctttggctggagccacaacagcgcggagatcaaccacgcgaaactgttcctgaccagctttaactggcagg
gtgaagtgacctgcctggcgcgtctgctgattaacgaggaaccggtttggatcaacctgattcgtgcgtacggtttcaccaagaaagcggtt
ctggagatcagcggcaagattaaacagcaactgccggtggcggagttcccgctggaagttagcagctttagcccgcagctgcaaatgccg
tttcagcaaagctatctggtggttaccccggtggttagccacgcgatgctggcgaagatccagcaactgaccaccgaccgtaaactgaactt
cgcgctggttgagcacagccgtccggcgaacgttggtgatctggcgagcagcgtgggtggcaacattcgtgttctgcgttactttccgaaga
cctatagcaaagcggtgaaccgtagcaaagttgcgaacaacgatatcgaaaaggcgttcaaaattcgtgcgctgctgagcagccagtttca
gcaagcgctgctggtgctggttggcatcaagcagttcaacaccctgcgtcaaaaacgtctggcgcgtgtggcggcgatccgtcaagtgcgt
gttagcctgcaactgtggctggacaacattctggaggcgaagaacaacgcgcagaaccaagtgtacccggaatgggttcgtcactatctgg
atcaaagcatcaccaactgcattagccagttcagcaacgttctgaacgaaagcctgggtaacctgagcaagctgaaacgttttgcgtaccac
ccgaacctgatgggcctgttcaaagcgcaactgaactatgtgtttacccactgcgcggcggagcaggaaatcctgaacgacgagcaaattg
tgtacgttcactgccaggacatgcgtgttttcgatgcggaagcgatggcgaacccgtatatccagggtatgccgagcctgaccgcgctgaa
cggcctggcgcacaacttcgagcgtaagctgaaaaactttattgatccgagcatcaagtgcattggtagcgcgatctacattgagaactatca
actgcacaccggcaaaccgctgccggaaccgagcaagctgaaacaggtggcgggtcgtagccacgttatccgtagcggcatcattgaca
agccgaaatgcgacattaccctggatctggtgttccgtctgtttgttccgaacaccgaactgctggataagctgaacagccaactgattaagc
cggcgctgccgagcagctttgcgggtggcaccatgcacccgccgagcctgtaccagaacattgactggtgccacgtgcacaccaagccg
agcgagctgtttaagaaactgaaggcgaaaagcagcaacggtagctggctgtatccgagcaagaaagtggttaaaagcttcgaacagctg
atcgacgcgctgaacagcaactttaacctgcgtccggcggcgattggcctggcggcgctggaggaaccggtgaagcgtgatgcggcgct
gcacgagtaccactgctatgcggaaccggttatcggtctgctggagtgcgtgagcaacaccagcgttaagtacgcgggcgcgaaacaatt
ctttcacgacgcgttctgggtgatggatgttcagaaggaaagcatgctgatgaagaaaagcaaatttgagtatgaataatgcagctgccgcgt
cacctgagctacacccgtagcctgagcccgagcaaggcggtgttcttttataaaaccccggagagcgacttcgaaccgctgcagatcgag
caaaacaaactggtgggtcagaagagcggttttggcgatgcgtaccagaagcaaaacgttgcgaaaaacctggcgccgcaggacctggc
gtttggtaacccgcaaaccattgatgtgtgctatgttccgccgaccgtgaacgaactgttctgccgttttagcctgcgtgttgaggcgaactgc
atcgaaccgcacgtgtgcgacgatccgaaggttatttactggctgaaacgtttctttgaaacctataagaaacacaacggtctgaacgaagtg
gcgacccgttacgcgaagaacatcctgatgggcaactggctgtggcgtaaccgtcagagcccgaacgttgacatcgagattctgaccgaa
cacgcggcgccgattgtggttgagggtgcgcagaagctgaaatggcaaggcaactggcagaacaaccaaaccgcgctgctgaccctga
gcgagagcatccaggaaggtctgagcaacccgcaaaactactgctatctggatatcaccgcgaagattaaaaacgcgttcagccaggaag
tgcacccgagccaaaagtttgtggacaacgttgaacagggtatgagcagcaaacagctggcgtatacccaagtgggcgataagaaagcg
gcgagcctgaacagccagaaggttggcgcggcgatccaaaccattgacgattggtacgaggaaggttataaaccgctgcgtacccatga
gtatggtgcggacaagcaaatcctggtggcgcaccgtaccccgaaaagccacagcgatttttatagcctgctgccgcgtatcgcgctgcac
attaagcacatggaaaaacacggtctggagcagagcgaacaaagcaacagcatccacttcattgcggcggttctgattaagggtggcctgt
ttcagcgtagcaaaggatgaagcgttactatttcaccatcacctacctgccgcaaagctgcgatgtgagcctgctggcgggtcgttgcatcg
gcattctgcacggtttcatgagcagccgtgagatcagcaacattggcgtgtgctttccgaaatggaacgagcagaccatcggtaacgaactg
gcgtttgttagcaccaacaagaaacaactgaccaacctgagccagcaaagctatttcgagatgatggcgcacgacaagctgtttggcctga
gcaaaattctggaagtgccggttaaccagagcgaagtgatgttcgttcgtaaccaaagcgtggcgaaggcgtttgttggtgaaaagcaacgt
cgtctgaaacgtgcgaagaaacgtgcggaggcgcgtggcgaagtgtacaacccggagtataagttcgaagcgaaagatatcggtcacttt
cacagcattccggtgagcagcaagggtaacggccagagctacgttctgcacatccaaaagaacgagaacgcggaaagcattaaaaacca
gttcaacaactatggctttgcgaccaaccaaattttcctgggcaccgtgccgagcctgaacaccctgctgtaaaaggagatataccatgtacc
gtcgtaagctgaaatatagccgtgttaagaacctgcacaaatttgcgagccagaagaacaaaagcacctgcctggtggagagcagcctgg
aattcgacgcgtgcttccactttgagttcagcccgccgatcgcggcgtttgaagcgcaaccgctgggttacgagtatgaattcgataaccgta
tttgccgttacaccccggactttctgctgacccacaccgatggcacccagaagttcatcgaggttaagccgcaaagcaaaattgcggacga
ggattttcgtgcgcgtttcatcgaaaagcaggcgattgcgaaacaagacggtcgtgatctgatcctggtgaccgacaagcagattcgtgttta
cccgaccctgaacaacctgaaactgctgcaccgttatagcggctttcagagcctgaccgagctgcaagcgagcgtgctggaactggttaag
cagtacggtagcatcaaagtgggccaactgattcgttatctgaaagttaccgcgggtgaactgctggcgaccgtgctgcgtctgctgagcct
gggccaactgttcgcggatctgaccaccaacgagatcagcattgaaaccgcgatctggagcaacaatgtttaataacgacctgttcgacgat
gagtttaaccagccgctgccgaaggcggaaaccaaactgccgcagaactataccaaggatctgcaagcgctgccggagaagatcaaaac
caccaccttcgcgaagctgaaatacattcaatggctggaggcgaacatccagggtggctggacccaaaagaacctggaaccgctgctgaa
actgatgccggacgttgagggtgaaaagaaaccgagctggcgtaccgcggcgcgttggtatagcgcgtacaccaacgcggataagaac
attatggcgctgatcccgagccaccagaagaaaggcaaccgtgaacgtgacaccaccaccgataagttct
SEQ ID NO. 7:
gtttctgtgacccgatcttttccggcagacagatttccagctgcctactcggcagttcacccatggtatatctccttttaaagtcgtg
acaccaccaccgataagttctcgtgacaccaccaccgataagttctttgagaaagcgctggaacgttacctggtgaaggagaaaccgagcg
ttgcgagcgcgtataagttctacaaagacctggtgatcattgaaaacgacagcgtggttgatagcgttctgaaaccgctgacctataaggcgt
ttaaaaaccgtattgacaacctgccgcagtatgaggttatgatcgcgcgttacggcaagcgtctggcggatattgcgtacaacaaggtggaa
ggccacaaacgtccgattcgtgtgctggagaaagttgaaatcgaccacaccccgctggatctgattctgctggacgatgagctgcacatcc
cgctgggtcgtccgaccctgaccatgctggttgacgtttatagccactgcatcgtgggctactatttcagctttagcgagccgagctacgatg
cggttcgtcgtgcgatgctgaacgcgatgaagccgaaaagcgaagtggcgaaactgtacccggacaccattaacgagtggaagtgcgcg
ggtaaaatcgaaaccctggtggttgataacggcgcggagttctggagcaacagcctggaactggcgtgcgaggaaatcggtattaacacc
cagtataacccggtggcgaagccgtggctgaaaccgttcgttgagcgtatgtttggcaccatcaacaccgaactgctggacccggttccgg
gcaagaccttcagcaacatcctgcaaaaacacgaatacaacccgaagaaagacgcgattatgcgtttcaccacctttatgcagctgtttcaca
agtgggtggttgatgtgtatcaccaagacgcggatagccgtttcaaatacattccgagccagctgtgggaccaaggctttaacaccctgccg
ccgaccatgctgagcgatgcggatctgcagcaactggatgtggttctgagcatcagcaaccaccgtgtgctgcgtaagggtggcattcgtct
ggagaacctgagctatgacagcaccgaactggcgaactaccgtaagcagttcagccacaaagtgagccaagaggttctgatcaaactgaa
cccggacgatattagctacatctatgtgtacctggacaagctggaacactatattaaagttccgtgcatcgatccgaacggttacacccagaa
cctgagcctgaaccaacacaagatcaacattcgtatccaccgtgactttattagcggtagcatcgataacgttggcctggcgaaggcgcgta
tgttcattcacaacaaaatccagaacgagtttgaggaactgaagaacgcgccgaaacacagcaaggtgaaaggtggcaaggcgctggcg
aaacaccagaacattagcagcgacagccaaaagagcatcacccacagcaaaccggtggaggcgaagaaagttaccccgaaagaacaa
ccgaccgatagctgggacgatttcatcagcgacctggatggtttttaattatgctgaccgacaagcagaaagaaaagctgaacgagttccgt
gatgtttttattgaatacccgatcattaccaccatcttcaacgactttgatcgtctgcgtctgggtaaaggcctgaccggcgagaagccgtgca
tgctgctgaacggtgacaccggcaccggtaaaaccgcgctgattaaacagtataaggaacgtcacctgccgcaattcatcaacggtgttatg
aaccacccggtgctggttagccgtattccgagcaacccgaccctggaaagcaccctggcggagctgctgaaagacctgggtcaagtggg
cagcaccgagcgtaagctgcgtattaacggcacccgtctgaccaccagcctgatcaaatgcctgaagacctgcggcaccgaactgatcatt
atcgatgagtttcaggaactgattgagcacaaccaaggcaagaaacgtcgtgaaattgcgaaccgtctgaaatacatcaacgacgaggcg
ggtgttagcattgtgctggttggcatgccgtgggcggaaaagatcgcggatgagccgcagtggagcagccgtctgctgatccgtcgtcaac
tgccgtatttcaaactgagcgagaacccgaagcactttgtgcagctgattatcggtctggcgaaccgtatgccgttcgcggaaaaaccgaac
ctgagcgagcaagcgaccgttttcaccctgtttagcctgagcaaaggctgcttccgtaccctgaagtactttctggacgatgcggtgctgtat
gcgctgatggacaacgcgaagaccctgaccaccaaacacctggtgaaggcgttcgaagttctgtttccggatgtgccgaacctgtttaccct
gccggttgcggagatcaccgcgagcgaggtggaacgttacagcctgtataagccggaaagcagccaggacgaggacccgttcattgcg
accaaatttaccgatcgtatgccgatcagccaactgctgcgtaagtaactcgagccgctgagcaataactagcataaccccttggggcctct
aaacgggtcttgaggggttttttgctgaaacctcaggcatttgagaagcacacggtcacactgcttccggtagtcaataaaccggtaaaccag
caatagacataagcggctatttaacgaccctgccctgaaccgacgacaagctgacgaccgggtctccgcaagtggcacttttcggggaaat
gtgcgcggaacccctatttgtttatttttctaaatacattcaaatatgtatccgctcatgaattaattcttagaaaaactcatcgagcatcaaa
tgaaactgcaatttattcatatcaggattatcaataccatatttttgaaaaagccgtttctgtaatgaaggagaaaactcaccgaggcagttcc
ataggatggcaagatcctggtatcggtctgcgattccgactcgtccaacatcaatacaacctattaatttcccctcgtcaaaaataaggttatc
aagtgagaaatcaccatgagtgacgactgaatccggtgagaatggcaaaagtttatgcatttctttccagacttgttcaacaggccagccatta
cgctcgtcatcaaaatcactcgcatcaaccaaaccgttattcattcgtgattgcgcctgagcgagacgaaatacgcggtcgctgttaaaaggac
aattacaaacaggaatcgaatgcaaccggcgcaggaacactgccagcgcatcaacaatattttcacctgaatcaggatattcttctaatacctg
gaatgctgttttcccggggatcgcagtggtgagtaaccatgcatcatcaggagtacggataaaatgcttgatggtcggaagaggcataaattcc
gtcagccagtttagtctgaccatctcatctgtaacatcattggcaacgctacctttgccatgtttcagaaacaactctggcgcatcgggcttcc
catacaatcgatagattgtcgcacctgattgcccgacattatcgcgagcccatttatacccatataaatcagcatccatgttggaatttaatcg
cggcctagagcaagacgtttcccgttgaatatggctcatactcttcctttttcaatattattgaagcatttatcagggttattgtctcatgagc
ggatacatatttgaatgtatttagaaaaataaacaaataggcatgctagcgcagaaacgtcctagaagatgccaggaggatacttagcagagag
acaataaggccggagcgaagccgtttttccataggctccgcccccctgacgaacatcacgaaatctgacgctcaaatcagtggtggcgaaaccc
gacaggactataaagataccaggcgtttccccctgatggctccctcttgcgctctcctgttcccgtcctgcggcgtccgtgttgtggtggaggc
tttacccaaatcaccacgtcccgttccgtgtagacagttcgctccaagctgggctgtgtgcaagaaccccccgttcagcccgactgctgcgcct
tatccggtaactatcatcttgagtccaacccggaaagacacgacaaaacgccactggcagcagccattggtaactgagaattagtggatttaga
tatcgagagtcttgaagtggtggcctaacagaggctacactgaaaggacagtatttggtatctgcgctccactaaagccagttaccaggttaa
gcagttccccaactgacttaaccttcgatcaaaccgcctccccaggcggttttttcgtttacagagcaggagattacgacgatcgtaaaagga
tctcaagaagatcctttacggattcccgacaccatcactctagatttcagtgcaatttatctcttcaaatgtagcacctgaagtcagccccata
cgatataagttgtaattctcatgttagtcatgccccgcgcccaccggaaggagctgactgggttgaaggctctcaagggcatcggtcgagatcc
cggtgcctaatgagtgagctaacttacattaattgcgttgcgctgatgaatcccctaatgatttttatcaaaatcattaaggttaccatcacgg
aaaaaggttatgctgcttttaagacccactttcacatttaagttgtttttctaatccgcatatgatcaattcaaggccgaataagaaggctggc
tctgcaccttggtgatcaaataattcgatagcttgtcgtaataatggcggcatactatcagtagtaggtgtttccctttcttctttagcgactt
gatgctcttgatcttccaatacgcaacctaaagtaaaatgccccacagcgctgagtgcatataatgcattctctagtgaaaaaccttgttggca
taaaaaggctaattgattttcgagagtttcatactgtttttctgtaggccgtgtacctaaatgtacttttgctccatcgcgatgacttagtaaa
gcacatctaaaacttttagcgttattacgtaaaaaatcttgccagctttccccttctaaagggcaaaagtgagtatggtgcctatctaacatct
caatggctaaggcgtcgagcaaagcccgcttattttttacatgccaatacaatgtaggctgctctacacctagcttctgggcgagtttacgggt
tgttaaaccttcgattccgacctcattaagcagctctaatgcgctgttaatcactttacttttatctaatctagacatcattaattcctaattt
ttgttgacactctatcattgatagagttattttaccactccctatcagtgatagagaaaagtgaactctagaaataattttgtttaactttaaa
aggagatataccatgggtgaactgccgagtaggcagctggaaatctgtctgccggaaaagatcgggtcacagaaac

The above four fragments were respectively recovered by gel extraction.

(2) Curing of template plasmids:

The four fragments obtained in the step (1), namely the target gene fragment, PD-vector, PQ-CbrB-1, and PQ-CbrB-2, were digested with Qcut-DPN1 to cure the template plasmids.

(3) Strains containing PD-empty plasmid and PQ-CbrB plasmid:

Using a homologous recombinase from ABclone, the fragments obtained in the step (2) after curing of template plasmids, namely the target gene fragment and PD-vector, PQ-CbrB-1 and PQ-CbrB-2, were respectively subjected to homologous recombination.

Since the PQ plasmid is relatively large, it was divided into two parts for separate PCR amplification. The two fragments PQ-CbrB-1 and PQ-CbrB-2 are both products of reverse PCR, each having a size of about 5,000 bp. The two groups of fragments, namely the target gene fragment and PD-vector, and PQ-CbrB-1 and PQ-CbrB-2, were then subjected to homologous recombination to form the PD-empty plasmid and the PQ-CbrB plasmid.

The two plasmids were electroporated into JM109 competent cells to generate strains JM109-PD and JM109-PQ.

(4) cultivation of strains:

The strains obtained in the step (3) were cultured for 1 hour, and then centrifuged at 5,000 rpm for 2 minutes. The JM109-PD strain was streaked onto solid LB plates containing chloramphenicol (Cm) at a final concentration of 33 μg/mL, while the JM109-PQ strain was streaked onto solid LB plates containing kanamycin (Kana) at a final concentration of 50 μg/mL. The plates were incubated for 12 hours until single colonies appeared.

(5) Sequencing verification

The single colonies of the JM109-PD strain obtained in the step (4) were subjected to colony PCR using PD-YZ-s and PD-YZ-x primers, followed by gel electrophoresis verification. A band size of 1,784 bp for the empty vector terminator sequence was identified as correct JM109-PD strain and sent for sequencing.

JM109-PQ could not be verified by colony PCR and required sequencing for verification. A sequencing primer for JM109-PQ was PQ-YZ. Strains verified as correct were preserved, cultured, and subjected to plasmid extraction using a Vazyme plasmid extraction kit.

PD-empty plasmid and PQ-CbrB plasmid were respectively obtained.

(6) The two plasmids extracted in the step (5) were simultaneously electroporated into three competent strains lys-1, lys-2, and lys-3, forming strains lys-1-PD-PQ, lys-2-PD-PQ, and lys-3-PD-PQ.

The above three strains lys-1-PD-PQ, lys-2-PD-PQ, and lys-3-PD-PQ were respectively streaked onto solid LB plates containing dual resistance of chloramphenicol (Cm) at a final concentration of 33 μg/mL and kanamycin (Kana) at a final concentration of 50 μg/mL for cultivation.

The three strains lys-1-PD-PQ, lys-2-PD-PQ, and lys-3-PD-PQ are collectively referred to as the lys-PD-PQ strains.

### 2. Induced Expression of Plasmids in the Lys-PD-PQ Strains

Subsequently, induction was performed on the two plasmids contained in the lys-PD-PQ strains, and a target gene (terminator) was inserted into the 32 bp target sequence (SEQ ID NO. 4) located upstream of the cbrB gene in the chassis strains lys-1, lys-2, and lys-3, thereby generating strains lys1.1 (CCTCC NO: M2019435AcbrB), lys-2.1 (NRRLB-12185ΔcbrB), and lys-3.1 (GDMCC NO. 1.318ΔcbrB), respectively.

Specific steps are as follows:

(1) The lys-PD-PQ strains obtained in the step 1 (lys-1-PD-PQ, lys-2-PD-PQ, lys-3-PD-PQ) were streaked onto solid LB plates supplemented with three antibiotics for induction. The antibiotics were chloramphenicol (Cm) at a final concentration of 33 μg/mL, kanamycin (Kana) at a final concentration of 50 μg/mL, and anhydrotetracycline hydrochloride at a final concentration of 1 mg/L, respectively.

(2) Single colonies obtained in the step (1) were subjected to colony PCR using verification primers PJY-YZ-S-1 and PJY-YZ-X-2 to verify insertion of the target gene into the target sequence.

Gel electrophoresis verification showed that a band size of 1,784 bp, indicating that the empty target gene had been inserted into the target locus. The strains subsequently verified as correct via sequencing (lys-PD-PQ strains) were then subjected to the next modification.

(3) The lys-PD-PQ strains (lys-1-PD-PQ, lys-2-PD-PQ, lys-3-PD-PQ) were prepared as electrocompetent cells, followed by electroporation with a Pcut plasmid (These plasmids are available from Addgene or Molecular Cloud. The website is: https://www.addgene.org. The catalog numbers are as follows: Addgene (Cat. Nos. 140622-140632) and Molecular Cloud (Cat. Nos. 101225-101235), used for curing PD and PQ plasmids), cultured for 3 h, and then streaked onto plates containing ampicillin (Amp) at a final concentration of 100 μg/mL and L-rhamnose at a final concentration of 10 mmol/L to induce the lys-PD-PQ strains, and cultured for 24 hours.

(4) The induced single colonies in the step (3) were streaked onto plates containing Kana at a concentration of 50 μg/mL, Cm at a concentration of 33 μg/mL, and Amp at a concentration of 100 μg/mL, respectively, and cultured for 24 hours. When no growth was observed on Kana and Cm plates but growth was observed on Amp plate, it indicated successful curing of PQ and PD plasmids. The correct strains were then cultured in LB medium.

(5) The strains obtained in the step (4) were streaked onto solid LB plates containing 10 μg/L sucrose for secondary induction to cure the Pcut plasmid, followed by cultivation for 24 hours.

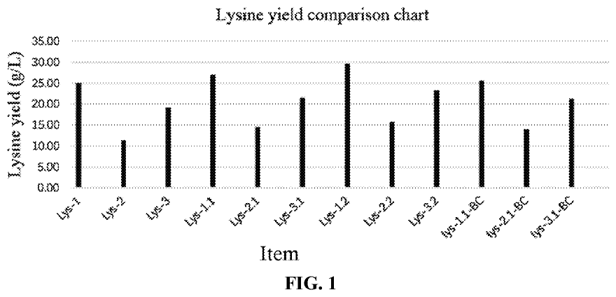

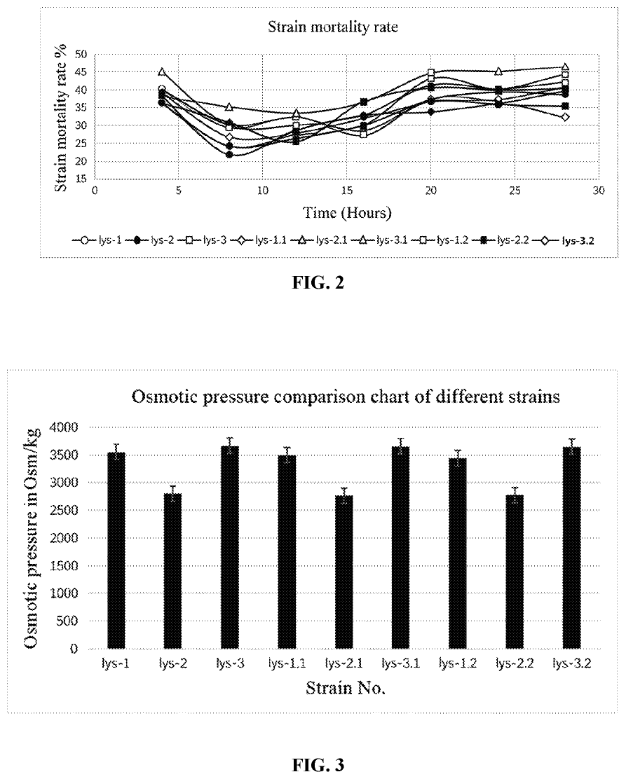

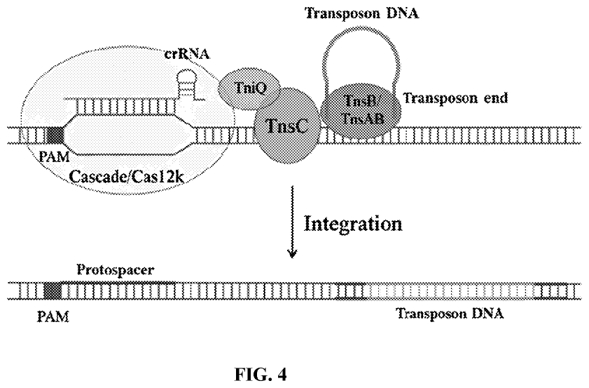

(6) The single colonies obtained in the step (5) were cultured on plates containing 100 g/mL Amp and on antibiotic-free LB solid medium for 24 hours. When no growth was observed on the Amp plate but growth was observed on the antibiotic-free plate, it indicated successful curing of the Pcut plasmid. The plasmid-free strains were cultured in liquid LB for 24 hours, thereby obtaining strains lys-1.1, lys-2.1, and lys-3.1. A schematic diagram of the genetic modification principle is illustrated in FIG. 1.

lys1.1 (CCTCC NO:M2019435ΔcbrB), lys2.1 (NRRLB-12185ΔcbrB), and lys3.1 (GDMCC NO. 1.318ΔcbrB) are collectively referred to as the first-generation lys strains.

### Example 2: Iterative Construction of Second-Generation Lys Strains from the First-Generation Lys Strains

The strains lys1.1 (CCTCC NO:M2019435ΔcbrB), lys2.1 (NRRLB-12185ΔcbrB) and lys3.1 (GDMCC NO. 1.318ΔcbrB) were used as chassis strains for genetic modifications. Using an MUCICAT gene editing method, a target gene (terminator) with a nucleotide sequence as shown in SEQ ID NO. 3 was used to replace a 32 bp target sequence (aaagtttgcaggtagttttcaggatgatgcca, SEQ ID NO. 5) located upstream of a cbrC gene in the L-lysine producing strains. The specific steps are as follows:

1. strains obtained after silencing the CbrC gene in the genome of the first-generation lys strains

(1) acquisition of different fragments:

Using a genome of the model E. coli strain MG1655 as a template, PCR amplification was performed with primers P1 and P2, and the amplified product was recovered to obtain a fragment of the target gene with a nucleotide sequence as shown in SEQ ID NO. 3. Using an original PD-GFP plasmid as a template, reverse PCR amplification of the plasmid was performed with primers P3 and P4 to obtain a vector fragment of the pDONOR plasmid: PD-vector.

Using the original PQ plasmid as a template, PCR amplification was performed with primers P9 P10, P11, and P12 to obtain a PQ-CbrC-1 fragment (SEQ ID NO. 8) and a PQ-CbrC-2 fragment (SEQ ID NO. 9);

SEQ ID NO. 8:
aaagtttgcaggtagttttcaggatgatgccagtgaactgccgagtaggcagctgaagttggatccgaattcgagctcgggag
aacttatcggtggtggtgtcacgaaagtttgcaggtagttttcaggatgatgccagtgaactgccgagtaggcagctgaagttggatccgaat
tcgagctcgggaagaaggagatatacatatgcactttctggtgcagaccaagagctacccggacgaggcgctggaaagctatctgctgcgt
ctggcgcgtgataacagctacaacggttatagcgagctggcggacatcctgtggcagtggctggcggaacaagataacgagctggaagg
tgcgctgccgctggcgctgagcaaggtggacgtttaccacgcgcgtcaggcgagcagcttccgtatccgtgcgctgaaactggtggcgca
actggcggacgttaacgcgggtgatattctggcgctggcgtggcgtcgtagcaacttcaagtttggcaacctggcggcggtgagccgtaac
gagctggcgatcccgctggaactgctgcgtaccgataacatcccggtttgcattaaatgcctgagcgagagcagccacattccgttttactg
gcacctgaagccgtataaagcgtgccacaagcacaaaagccagctgatcacccgttgcaaggagtgctacgacctgattgattatcgtgcg
agcgaggcgtttctggaatgcgtttgcggttgcaaaatcaccaacagcgaacaactgaacgacgcggatttcaagatcgcgattgcgctgg
cgagcagcaacagccagaaaatcgtgggcctgattagctggttcgcgaaggtgaaacaactggacgttagcgacgcggatttcaactgcg
cgtttgttgattacttcaacacctggccggagagcctgaccaccgaactggacctgctgaccaacaacgcgcgtctgaagcagctgaaccc
gtttaacaagaccaaattcagcagcgtgtacggtgacctgatccgtgatggccaaattgcggcgaccagcaaccgtaagaacaaagttatc
gacgagatcattagctattttgtggaactggttgatagcaacccgaaggcgaaacacccgaacattggtgacctgctgctgtgcaccttcgat
gcggcggtgctgctgaacaccaccaccgagcaggtttaccgtctgcaccaagaagcgtttctgaactgcgcgtatagccagaagaaacac
gaacaactgcgtgcggatagccacgtgttctatctgcgtcaggttatcgagctgcagcaagcgtttgcggcggaaaaaccgctgaccaaga
aacaattcattgcgccgtggtaacttatgaacctgcaggatgcgctggcgattgagccgctgaaggaaaaaaccaccgcgctgcgtaagct
gttcgtgccgtacaccagccacgttgaggtggatggttttgaggaactggcgctgaccgtgctgatcaacctggtttataagcgtagcgaaat
tgacgatctgaccagcgcgcgtaccgcgaaaagcgtgctgcgtgacgaggttctgctgagcaagtgcatcaacgaagtgaaatggttcca
cacccacaacctgaagtacccggacatccgtgttagccaccaacgtctgattagcgaggtggttagcgaagatatcgcgggtatttgcagc
cgtagcctgccgctgagctttggctggagccacaacagcgcggagatcaaccacgcgaaactgttcctgaccagctttaactggcagggt
gaagtgacctgcctggcgcgtctgctgattaacgaggaaccggtttggatcaacctgattcgtgcgtacggtttcaccaagaaagcggttct
ggagatcagcggcaagattaaacagcaactgccggtggcggagttcccgctggaagttagcagctttagcccgcagctgcaaatgccgttt
cagcaaagctatctggtggttaccccggtggttagccacgcgatgctggcgaagatccagcaactgaccaccgaccgtaaactgaacttcg
cgctggttgagcacagccgtccggcgaacgttggtgatctggcgagcagcgtgggtggcaacattcgtgttctgcgttactttccgaagacc
tatagcaaagcggtgaaccgtagcaaagttgcgaacaacgatatcgaaaaggcgttcaaaattcgtgcgctgctgagcagccagtttcagc
aagcgctgctggtgctggttggcatcaagcagttcaacaccctgcgtcaaaaacgtctggcgcgtgtggcggcgatccgtcaagtgcgtgt
tagcctgcaactgtggctggacaacattctggaggcgaagaacaacgcgcagaaccaagtgtacccggaatgggttcgtcactatctggat
caaagcatcaccaactgcattagccagttcagcaacgttctgaacgaaagcctgggtaacctgagcaagctgaaacgttttgcgtaccaccc
gaacctgatgggcctgttcaaagcgcaactgaactatgtgtttacccactgcgcggcggagcaggaaatcctgaacgacgagcaaattgtg
tacgttcactgccaggacatgcgtgttttcgatgcggaagcgatggcgaacccgtatatccagggtatgccgagcctgaccgcgctgaacg
gcctggcgcacaacttcgagcgtaagctgaaaaactttattgatccgagcatcaagtgcattggtagcgcgatctacattgagaactatcaac
tgcacaccggcaaaccgctgccggaaccgagcaagctgaaacaggtggcgggtcgtagccacgttatccgtagcggcatcattgacaag
ccgaaatgcgacattaccctggatctggtgttccgtctgtttgttccgaacaccgaactgctggataagctgaacagccaactgattaagccg
gcgctgccgagcagctttgcgggggcaccatgcacccgccgagcctgtaccagaacattgactggtgccacgtgcacaccaagccgag
cgagctgtttaagaaactgaaggcgaaaagcagcaacggtagctggctgtatccgagcaagaaagtggttaaaagcttcgaacagctgat
cgacgcgctgaacagcaactttaacctgcgtccggcggcgattggcctggcggcgctggaggaaccggtgaagcgtgatgcggcgctg
cacgagtaccactgctatgcggaaccggttatcggtctgctggagtgcgtgagcaacaccagcgttaagtacgcgggcgcgaaacaattct
ttcacgacgcgttctgggtgatggatgttcagaaggaaagcatgctgatgaagaaaagcaaatttgagtatgaataatgcagctgccgcgtc
acctgagctacacccgtagcctgagcccgagcaaggcggtgttcttttataaaaccccggagagcgacttcgaaccgctgcagatcgagc
aaaacaaactggtgggtcagaagagcggttttggcgatgcgtaccagaagcaaaacgttgcgaaaaacctggcgccgcaggacctggcg
tttggtaacccgcaaaccattgatgtgtgctatgttccgccgaccgtgaacgaactgttctgccgttttagcctgcgtgttgaggcgaactgca
tcgaaccgcacgtgtgcgacgatccgaaggttatttactggctgaaacgtttctttgaaacctataagaaacacaacggtctgaacgaagtgg
cgacccgttacgcgaagaacatcctgatgggcaactggctgtggcgtaaccgtcagagcccgaacgttgacatcgagattctgaccgaac
acgcggcgccgattgtggttgagggtgcgcagaagctgaaatggcaaggcaactggcagaacaaccaaaccgcgctgctgaccctgag
cgagagcatccaggaaggtctgagcaacccgcaaaactactgctatctggatatcaccgcgaagattaaaaacgcgttcagccaggaagt
gcacccgagccaaaagtttgtggacaacgttgaacagggtatgagcagcaaacagctggcgtatacccaagtgggcgataa gaaagcgg
cgagcctgaacagccagaaggttggcgcggcgatccaaaccattgacgattggtacgaggaaggttataaaccgctgcgtacccatgagt
atggtgcggacaagcaaatcctggtggcgcaccgtaccccgaaaagccacagcgatttttatagcctgctgccgcgtatcgcgctgcacat
taagcacatggaaaaacacggtctggagcagagcgaacaaagcaacagcatccacttcattgcggcggttctgattaagggtggcctgttt
cagcgtagcaaaggatgaagcgttactatttcaccatcacctacctgccgcaaagctgcgatgtgagcctgctggcgggtcgttgcatcggc
attctgcacggtttcatgagcagccgtgagatcagcaacattggcgtgtgctttccgaaatggaacgagcagaccatcggtaacgaactggc
gtttgttagcaccaacaagaaacaactgaccaacctgagccagcaaagctatttcgagatgatggcgcacgacaagctgtttggcctgagc
aaaattctggaagtgccggttaaccagagcgaagtgatgttcgttcgtaaccaaagcgtggcgaaggcgtttgttggtgaaaagcaacgtcg
tctgaaacgtgcgaagaaacgtgcggaggcgcgtggcgaagtgtacaacccggagtataagttcgaagcgaaagatatcggtcactttca
cagcattccggtgagcagcaagggtaacggccagagctacgttctgcacatccaaaagaacgagaacgcggaaagcattaaaaaccagt
tcaacaactatggctttgcgaccaaccaaattttcctgggcaccgtgccgagcctgaacaccctgctgtaaaaggagatataccatgtaccgt
cgtaagctgaaatatagccgtgttaagaacctgcacaaatttgcgagccagaagaacaaaagcacctgcctggtggagagcagcctggaa
ttcgacgcgtgcttccactttgagttcagcccgccgatcgcggcgtttgaagcgcaaccgctgggttacgagtatgaattcgataaccgtattt
gccgttacaccccggactttctgctgacccacaccgatggcacccagaagttcatcgaggttaagccgcaaagcaaaattgcggacgagg
attttcgtgcgcgtttcatcgaaaagcaggcgattgcgaaacaagacggtcgtgatctgatcctggtgaccgacaagcagattcgtgtttacc
cgaccctgaacaacctgaaactgctgcaccgttatagcggctttcagagcctgaccgagctgcaagcgagcgtgctggaactggttaagca
gtacggtagcatcaaagtgggccaactgattcgttatctgaaagttaccgcgggtgaactgctggcgaccgtgctgcgtctgctgagcctgg
gccaactgttcgcggatctgaccaccaacgagatcagcattgaaaccgcgatctggagcaacaatgtttaataacgacctgttcgacgatga
gtttaaccagccgctgccgaaggcggaaaccaaactgccgcagaactataccaaggatctgcaagcgctgccggagaagatcaaaacca
ccaccttcgcgaagctgaaatacattcaatggctggaggcgaacatccagggtggctggacccaaaagaacctggaaccgctgctgaaac
tgatgccggacgttgagggtgaaaagaaaccgagctggcgtaccgcggcgcgttggtatagcgcgtacaccaacgcggataagaacatt
atggcgctgatcccgagccaccagaagaaaggcaaccgtgaacgtgacaccaccaccgataagttct
SEQ ID NO. 9:
tggcatcatcctgaaaactacctgcaaactttatttccagctgcctactcggcagttcacccatggtatatctccttttaaagtcgtg
acaccaccaccgataagttctcgtgacaccaccaccgataagttctttgagaaagcgctggaacgttacctggtgaaggagaaaccgagcg
ttgcgagcgcgtataagttctacaaagacctggtgatcattgaaaacgacagcgtggttgatagcgttctgaaaccgctgacctataaggcgt
ttaaaaaccgtattgacaacctgccgcagtatgaggttatgatcgcgcgttacggcaagcgtctggcggatattgcgtacaacaaggtggaa
ggccacaaacgtccgattcgtgtgctggagaaagttgaaatcgaccacaccccgctggatctgattctgctggacgatgagctgcacatcc
cgctgggtcgtccgaccctgaccatgctggttgacgtttatagccactgcatcgtgggctactatttcagctttagcgagccgagctacgatg
cggttcgtcgtgcgatgctgaacgcgatgaagccgaaaagcgaagtggcgaaactgtacccggacaccattaacgagtggaagtgcgcg
ggtaaaatcgaaaccctggtggttgataacggcgcggagttctggagcaacagcctggaactggcgtgcgaggaaatcggtattaacacc
cagtataacccggtggcgaagccgtggctgaaaccgttcgttgagcgtatgtttggcaccatcaacaccgaactgctggacccggttccgg
gcaagaccttcagcaacatcctgcaaaaacacgaatacaacccgaagaaagacgcgattatgcgtttcaccacctttatgcagctgtttcaca
agtgggtggttgatgtgtatcaccaagacgcggatagccgtttcaaatacattccgagccagctgtgggaccaaggctttaacaccctgccg
ccgaccatgctgagcgatgcggatctgcagcaactggatgtggttctgagcatcagcaaccaccgtgtgctgcgtaagggtggcattcgtct
ggagaacctgagctatgacagcaccgaactggcgaactaccgtaagcagttcagccacaaagtgagccaagaggttctgatcaaactgaa
cccggacgatattagctacatctatgtgtacctggacaagctggaacactatattaaagttccgtgcatcgatccgaacggttacacccagaa
cctgagcctgaaccaacacaagatcaacattcgtatccaccgtgactttattagcggtagcatcgataacgttggcctggcgaaggcgcgta
tgttcattcacaacaaaatccagaacgagtttgaggaactgaagaacgcgccgaaacacagcaaggtgaaaggtggcaaggcgctggcg
aaacaccagaacattagcagcgacagccaaaagagcatcacccacagcaaaccggtggaggcgaagaaagttaccccgaaagaacaa
ccgaccgatagctgggacgatttcatcagcgacctggatggtttttaattatgctgaccgacaagcagaaagaaaagctgaacgagttccgt
gatgtttttattgaatacccgatcattaccaccatcttcaacgactttgatcgtctgcgtctgggtaaaggcctgaccggcgagaagccgtgca
tgctgctgaacggtgacaccggcaccggtaaaaccgcgctgattaaacagtataaggaacgtcacctgccgcaattcatcaacggtgttatg
aaccacccggtgctggttagccgtattccgagcaacccgaccctggaaagcaccctggcggagctgctgaaagacctgggtcaagtggg
cagcaccgagcgtaagctgcgtattaacggcacccgtctgaccaccagcctgatcaaatgcctgaagacctgcggcaccgaactgatcatt
atcgatgagtttcaggaactgattgagcacaaccaaggcaagaaacgtcgtgaaattgcgaaccgtctgaaatacatcaacgacgaggcg
ggtgttagcattgtgctggttggcatgccgtgggcggaaaagatcgcggatgagccgcagtggagcagccgtctgctgatccgtcgtcaac
tgccgtatttcaaactgagcgagaacccgaagcactttgtgcagctgattatcggtctggcgaaccgtatgccgttcgcggaaaaaccgaac
ctgagcgagcaagcgaccgttttcaccctgtttagcctgagcaaaggctgcttccgtaccctgaagtactttctggacgatgcggtgctgtat
gcgctgatggacaacgcgaagaccctgaccaccaaacacctggtgaaggcgttcgaagttctgtttccggatgtgccgaacctgtttaccct
gccggttgcggagatcaccgcgagcgaggtggaacgttacagcctgtataagccggaaagcagccaggacgaggacccgttcattgcg
accaaatttaccgatcgtatgccgatcagccaactgctgcgtaagtaactcgagccgctgagcaataactagcataaccccttggggcctct
aaacgggtcttgaggggttttttgctgaaacctcaggcatttgagaagcacacggtcacactgcttccggtagtcaataaaccggtaaaccag
caatagacataagcggctatttaacgaccctgccctgaaccgacgacaagctgacgaccgggtctccgcaagtggcacttttcggggaaat
gtgcgcggaacccctatttgtttatttttctaaatacattcaaatatgtatccgctcatgaattaattcttagaaaaactcatcgagcatcaaa
tgaaactgcaatttattcatatcaggattatcaataccatatttttgaaaaagccgtttctgtaatgaaggagaaaactcaccgaggcagttcc
ataggatggcaagatcctggtatcggtctgcgattccgactcgtccaacatcaatacaacctattaatttcccctcgtcaaaaataaggttatc
aagtgagaaatcaccatgagtgacgactgaatccggtgagaatggcaaaagtttatgcatttctttccagacttgttcaacaggccagccatta
cgctcgtcatcaaaatcactcgcatcaaccaaaccgttattcattcgtgattgcgcctgagcgagacgaaatacgcggtcgctgttaaaaggac
aattacaaacaggaatcgaatgcaaccggcgcaggaacactgccagcgcatcaacaatattttcacctgaatcaggatattcttctaatacctg
gaatgctgttttcccggggatcgcagtggtgagtaaccatgcatcatcaggagtacggataaaatgcttgatggtcggaagaggcataaattc
cgtcagccagtttagtctgaccatctcatctgtaacatcattggcaacgctacctttgccatgtttcagaaacaactctggcgcatcgggcttc
ccatacaatcgatagattgtcgcacctgattgcccgacattatcgcgagcccatttatacccatataaatcagcatccatgttggaatttaatc
gcggcctagagcaagacgtttcccgttgaatatggctcatactcttcctttttcaatattattgaagcatttatcagggttattgtctcatgag
cggatacatatttgaatgtatttagaaaaataaacaaataggcatgctagcgcagaaacgtcctagaagatgccaggaggatacttagcagaga
gacaataaggccggagcgaagccgtttttccataggctccgcccccctgacgaacatcacgaaatctgacgctcaaatcagtggtggcgaaacc
cgacaggactataaagataccaggcgtttccccctgatggctccctcttgcgctctcctgttcccgtcctgcggcgtccgtgttgtggtggagg
ctttacccaaatcaccacgtcccgttccgtgtagacagttcgctccaagctgggctgtgtgcaagaaccccccgttcagcccgactgctgcgcc
ttatccggtaactatcatcttgagtccaacccggaaagacacgacaaaacgccactggcagcagccattggtaactgagaattagtggatttag
atatcgagagtcttgaagtggtggcctaacagaggctacactgaaaggacagtatttggtatctgcgctccactaaagccagttaccaggttaa
gcagttccccaactgacttaaccttcgatcaaaccgcctccccaggcggttttttcgtttacagagcaggagattacgacgatcgtaaaagga
tctcaagaagatcctttacggattcccgacaccatcactctagatttcagtgcaatttatctcttcaaatgtagcacctgaagtcagccccata
cgatataagttgtaattctcatgttagtcatgccccgcgcccaccggaaggagctgactgggttgaaggctctcaagggcatcggtcgagatcc
cggtgcctaatgagtgagctaacttacattaattgcgttgcgctgatgaatcccctaatgatttttatcaaaatcattaaggttaccatcacgg
aaaaaggttatgctgcttttaagacccactttcacatttaagttgtttttctaatccgcatatgatcaattcaaggccgaataagaaggctgg
ctctgcaccttggtgatcaaataattcgatagcttgtcgtaataatggcggcatactatcagtagtaggtgtttccctttcttctttagcgact
tgatgctcttgatcttccaatacgcaacctaaagtaaaatgccccacagcgctgagtgcatataatgcattctctagtgaaaaaccttgttggc
ataaaaaggctaattgattttcgagagtttcatactgtttttctgtaggccgtgtacctaaatgtacttttgctccatcgcgatgacttagtaa
agcacatctaaaacttttagcgttattacgtaaaaaatcttgccagctttccccttctaaagggcaaaagtgagtatggtgcctatctaacatc
tcaatggctaaggcgtcgagcaaagcccgcttattttttacatgccaatacaatgtaggctgctctacacctagcttctgggcgagtttacggg
ttgttaaaccttcgattccgacctcattaagcagctctaatgcgctgttaatcactttacttttatctaatctagacatcattaattcctaatt
tttgttgacactctatcattgatagagttattttaccactccctatcagtgatagagaaaagtgaactctagaaataattttgtttaactttaa
aaggagatataccatgggtgaactgccgagtaggcagctggaaataaagtttgcaggtagttttcaggatgatgcca

The above four fragments were respectively recovered by gel extraction.

(2) curing of template plasmids:

The four fragments obtained in the step (1), namely the target gene fragment, PD-vector, PQ-CbrC-1, and PQ-CbrC-2, were digested with Qcut-DPN1 to cure the template plasmids.

(3) Strains containing PD-Empty plasmid and PQ-CbrC plasmid:

Using a homologous recombinase from ABclone, the fragments obtained in the step (2) after curing of template plasmids, namely the target gene fragment and PD-vector, and PQ-CbrC-1 and PQ-CbrC-2, were respectively subjected to homologous recombination. Since the PQ plasmid is relatively large, it was divided into two parts for separate PCR amplification. The two fragments PQ-CbrC-1 and PQ-CbrC-2 are both products of reverse PCR, each having a size of about 5,000 bp. The two groups of fragments, namely the target gene fragment and PD-vector, and PQ-CbrC-1 and PQ-CbrC-2, were then subjected to homologous recombination to form the PD-empty plasmid and the PQ-CbrC plasmid. PD-empty plasmid and PQ-CbrC plasmid were respectively obtained.

The two plasmids were electroporated into JM109 competent cells to generate strains JM109-PD and JM109-PQ.

(4) cultivation of strains:

The strains obtained in the step (3) were cultured for 1 hour, and then centrifuged at 5,000 rpm for 2 minutes. The JM109-PD strain was streaked onto solid LB plates containing chloramphenicol (Cm) at a final concentration of 33 μg/mL, while the JM109-PQ strain was streaked onto solid LB plates containing kanamycin (Kana) at a final concentration of 50 μg/mL.

The plates were incubated for 12 hours until single colonies appeared.

(5) Sequencing verification

The single colonies of the JM109-PD strain obtained in the step (4) were subjected to colony PCR using PD-YZ-s and PD-YZ-x primers, followed by gel electrophoresis verification. A band size of 1,784 bp was identified as correct JM109-PD strain and sent for sequencing.

JM109-PQ could not be verified by colony PCR and required sequencing for verification. A sequencing primer for JM109-PQ was PQ-YZ. Strains verified as correct were preserved, cultured, and subjected to plasmid extraction using a Vazyme plasmid extraction kit.

PD-empty plasmid and PQ-CbrC plasmid were respectively obtained.

(6) Iterative construction of the second-generation lys strains based on the first-generation lys strains by knocking out the colicin E2 resistance protein CbrC: the two plasmids extracted in the step (5) were simultaneously electroporated into the competent cells of the first-generation lys strains to form strains lys-x.1-PD-PQ. The recipient strains for electroporation were transitioned from the lys chassis strains to the first-generation lys strains, thereby completing an iterative operation.

Specifically, the two plasmids extracted in the step (5) were simultaneously electroporated into the competent cells of the lys first-generation strains (lys1.1 (CCTCC NO:M2019435ΔcbrB), lys2.1(NRRLB-12185ΔcbrB), lys3.1 (GDMCC NO. 1.318ΔcbrB)) to form lys-x.1-PD-PQ strains.

The lys-x.1-PD-PQ strains were streaked onto solid LB plates containing dual resistance of chloramphenicol (Cm) at a final concentration of 33 μg/mL and kanamycin (Kana) at a final concentration of 50 μg/mL. Upon the appearance of single colonies, well-grown strains were selected for cultivation.

(7) Following the method described in Example 1, primers PJY-YZ-S-3 and PJY-YZ-X-4 were used to verify insertion of the empty vector fragment into the cbrC gene. The induction and plasmid curing process described in Example 2 was repeated. After plasmid curing, strains with silenced cbrB gene and the cbrC gene in the genome of the first-generation lys strains were obtained, which were designated as second-generation lys strains.

lys-1.2 (CCTCC NO: M2019435ΔcbrBΔcbrC), lys-2.2 (NRRLB-12185ΔcbrBΔcbrC), and lys-3.2(GDMCC NO. 1.318ΔcbrBΔcbrC) were prepared, respectively.

2. The specific implementation method is the same as that described in Example 1 and the step 1, except that: lys-1: E. coli CCTCC NO: M2019435; lys-2: E. coli NRRLB-12185; lys-3: E. coli GDMCC NO. 1.318 were used as chassis strains for independent genetic modifications. Using an MUCICAT gene editing method, a target gene (terminator) with a nucleotide sequence as shown in SEQ ID NO. 3 was used to replace a 32 bp target sequence (SEQ ID NO. 5) located upstream of a cbrC gene in the L-lysine producing strains.

lys-1.1-1 (CCTCC NO:M2019435ΔcbrC), lys-2.2-1 (NRRLB-12185 ΔcbrC), and lys-3.2-1 (GDMCC NO. 1.318 ΔcbrC) were prepared, respectively.

### Example 3: Comparative Fermentation Experiments of Different Strains Obtained by The Present Disclosure

1. In addition to the three generations of strains mentioned above, research on a series of strains derived from the three chassis strains lys-1, lys-2, and lys-3 in which only the cbrC gene was silenced was also conducted. They were designated as lys-1.1-1 (CCTCC NO:M2019435ΔcbrC), lys-2.2-1 (NRRLB-12185 ΔcbrC), and lys-3.2-1 (GDMCC NO. 1.318 ΔcbrC), collectively referred to as first-generation lys strains-1. The three chassis strains, first-generation lys strains, first-generation lys strains-1, and second-generation lys strains were respectively subjected to shake-flask fermentation experiments to obtain fermentation broths. A L-lysine content in each fermentation broth was measured.

The specific steps are as follows:

(1) Preparation of seed culture

First, 100 μL of strains from a glycerol stock were inoculated into 30 mL of LB medium and cultured at 37° C. for 24 hours, the culture was then streaked onto slant test tubes and incubated at 37° C. for 16-24 hours, and the slants were subsequently washed with 10 mL of sterile water to obtain a slant eluate.

2.5 mL (with an inoculum size of 2.5%) of the slant eluate was taken and inoculated into a primary seed medium (100 mL) and cultured at 37° C. with shaking at 180 rpm for 7-8 hours. When an OD₅₆₂ of the primary seed reached 4-5, 5 mL of the culture (with an inoculum size of 10% v/v) was transferred into a secondary seed medium (50 mL) and cultured at 37° C. with shaking at 180 rpm for 7-8 hours. When an OD₅₆₂ reached 16-17, the culture was inoculated into a fermentation medium with an inoculum size of 20% (v/v). Compositions of the seed media at each stage were as described above.

(2) Fermentation for L-lysine production

1) Compositions of a fermentation medium (g/L) used during fermentation were as follows:

Glucose 30 g/L, 85% H₃PO₄ 0.2 mL, potassium chloride 0.5 g/L, beet molasses 18 mL/L, betaine hydrochloride 1.5 g/L, MgSO₄·7H₂O 3.29 g/L, FeSO₄·7H₂O 49.4 mg/L, MnSO₄·H₂O 35.8 mg/L, ZnSO₄·7H₂O 152.9 mg/L, CuSO₄·5H₂O 120 mg/L, L-threonine 300 mg/L, corn steep liquor powder 7.4 g/L, vitamin B1 60 mg/L, nicotinamide 10 mg/L, biotin 0.6 mg/L;

2) The shake-flask fermentation conditions were as follows: an inoculum size of 20% (v/v); culture temperature 37° C.; shaker speed 180 rpm; pH control: no adjustment during 0-10 h, and pH maintained at 6-7 after 10 hours; shaker model: HYL-C³ combined shaker; fermentation duration: 30-42 hours.

2. After fermentation, an L-lysine content in the fermentation broth was determined using the ninhydrin colorimetric method. Experimental results of the fermentation of the three chassis strains, first-generation lys strains, first-generation strains-1, and second-generation lys strains are shown in Table 2.

TABLE 2
OD₅₆₂ and pH before fermentation, and experimental results after fermentation

| | Shake flask seed culture indicators | | | | | | |
|---|---|---|---|---|---|---|---|
| | Mean OD₅₆₂ | | | Mean OD₅₆₂ | Mean | Mean | |
| | (before | pH (before | Fermentation | (after | residual | L-lysine | Conversion |
| S/N | fermentation) | fermentation) | duration | fermentation) | sugar (g/L) | (g/L) | rate (%) |
| Lys-1 (CCTCC NO: M2019435) | 2.75 | 6.66 | 30 hours | 16.8 | 0.7 | 22.53 | 50.78% |
| Lys-2 (NRRLB-12185) | 2.05 | 6.74 | | 17.4 | 0.7 | 11.4 | 23.12% |
| Lys-3 (GDMCC NO. 1.318) | 2.28 | 6.73 | | 16.8 | 1 | 19.23 | 39.25% |
| Lys-1.1 (CCTCC | 1.7 | 6.92 | | 18.8 | 0.7 | 27 | 54.77% |
| NO: M2019435ΔcbrB) | | | | | | | |
| Lys-2.1 (NRRLB-12185ΔcbrB) | 2 | 6.82 | | 16.4 | 1 | 14.57 | 29.73% |
| Lys-3.1 (GDMCC NO. | 2.15 | 6.8 | | 17.1 | 0.7 | 21.6 | 43.81% |
| 1.318ΔcbrB) | | | | | | | |
| lys-1.1-1 (CCTCC | 2.03 | 6.6 | | 16.3 | 0.33 | 25.53 | 51.41% |
| NO: M2019435ΔcbrC) | | | | | | | |
| lys-2.1-1 (NRRLB- | 2.14 | 6.7 | | 17.4 | 1 | 14.03 | 28.64% |
| 12185ΔcbrC) | | | | | | | |
| lys-3.1-1 (GDMCC NO. | 2.31 | 6.76 | | 16.2 | 0.33 | 21.23 | 42.75% |
| 1.318ΔcbrC) | | | | | | | |
| Lys-1.2 (CCTCC | 2.15 | 6.75 | | 16.1 | 0.3 | 29.63 | 59.62% |
| NO: M2019435ΔcbrBΔcbrC) | | | | | | | |
| Lys-2.2 (NRRLB- | 2.15 | 6.76 | | 17.8 | 1 | 15.83 | 32.31% |
| 12185ΔcbrBΔcbrC) | | | | | | | |
| Lys-3.2 (GDMCC NO. | 2.4 | 6.86 | | 13.2 | 1 | 23.33 | 47.61% |
| 1.318ΔcbrBΔcbrC) | | | | | | | |

The results indicate that, compared with the L-lysine chassis strains lys-1, lys-2, and lys-3, the lysine yields of strains lys-1.1, lys-2.1, and lys-3.1 were significantly increased. In addition, lys-1.2, lys-2.2, and lys-3.2 could further significantly improve the yield of L-lysine compared with the former three strains (lys-1, lys-2 and lys-3). That is, based on the E. coli chassis strains, knockout of CbrB and CbrC can improve the yield and conversion rate of L-lysine.

### Example 4: Effect of Silencing Other Genes on Strains

The specific implementation method is the same as that described in Example 1, except that the chassis strain was replaced with lys-1 (E. coli CCTCC NO: M2019435). Following the method as described in Example 1, the MUCICAT gene editing method was used to replace the 32 bp target sequence (Table 3) located upstream of the sthA, AmtB, pck, or sucCD genes in the L-lysine producing strain with the target gene (terminator) whose nucleotide sequence is shown as SEQ ID NO. 3.

The procedures for obtaining strains with different genes silenced in the genome of lys-1 was the same as those described in the step 1, except that the silenced genes were replaced with sthA, AmtB, pck and sucCD, respectively; that is, the 32 bp sequence shown in SEQ ID NO. 10 in the original plasmid pQCascade was replaced with the 32 bp target sequences of sthA, AmtB, pck, and sucCD, respectively, to perform knockout of these genes. The 32 bp target sequences located upstream of the sthA, AmtB, pck, or sucCD genes in the L-lysine producing strain were replaced with the target gene (terminator) as shown in SEQ ID NO. 3. The specific target sequences are shown in Table 3.

SEQ ID NO. 10:
Tggatgctttgcgtgcaaaggaacctgaactc

New strains lys-1-ΔsthA, lys-1-ΔAmtB, lys-1-Δpck, and lys-1-ΔsucCD were constructed.

TABLE 3
Target sequences involved

| Gene name | 32 bp target sequence |
|---|---|
| sthA | tcatccaacagatgttgatttcacccatccac (SEQ ID NO. 34) |
| AmtB | ttctcagctgtgttgattttcgtggtggtatg (SEQ ID NO. 35) |
| pck | aaaagataagtatatcgtccgtgacgatacca (SEQ ID NO. 36) |
| sucCD | tgaagaagtggttattgcgcgttgccccctgg (SEQ ID NO. 37) |

Fermentation was carried out according to the method as described in Example 3. The testing results are shown in Table 4.

TABLE 4
Shake-flask results of strains

| | | | Yield of | Sugar | Conver- |
|---|---|---|---|---|---|
| | | | L-lysine | consump- | sion rate |
| Strain NO. | Genotype | OD₅₆₂ | (g/L) | tion (g) | (%) |
| Lys-1 | Original | 16.8 | 22.53 | 2.219 | 50.78 |
| | strain | | | | |
| Lys-1-ΔsthA | ΔsthA | 18.9 | 21.3 | 2.175 | 49.0 |
| (gene ID: | | | | | |
| 948461) | | | | | |
| lys-1-ΔsucCD | ΔsucC/D | 11.3 | 2.4 | 1.05 | 11.4 |
| (ID: 945312 | | | | | |
| and 945314) | | | | | |
| lys-1-Δpck | Δpck | 6.9 | 2.8 | 0.85 | 16.4 |
| (ID: 945667) | | | | | |
| lys-1-ΔAmtB | ΔAmtB | 17.9 | 20 | 2.15 | 46.5 |
| (ID: 945084) | | | | | |

Although the yield of strains did not show a significant increase, this modification method has broad applicability and is suitable for the silencing of various genes.

### Example 5: Comparison of Mortality Rates of Three Chassis Strains, First-Generation Lys Strains, and Second-Generation Lys Strains

The mortality rates of three chassis strains, first-generation lys strains, and second-generation lys strains were tested. The instrument used for testing was a flow cytometer (Model: Attune CytPix). The specific steps are as follows:

1. First, 100 μL of strains from a glycerol stock were inoculated into 30 mL of LB medium and cultured at 37° C. for 24 hours, the culture was then streaked onto slant test tubes and incubated at 37° C. for 16-24 hours, and the slants were subsequently washed with 10 mL of sterile water to obtain a slant eluate.

2.5 mL (with an inoculum size of 2.5%) of the slant eluate was taken and inoculated into a primary seed medium (100 mL) and cultured at 37° C. with shaking at 180 rpm for 7-8 hours. When an OD₅₆₂ of the primary seed medium reached 4-5, 5 mL of the culture (with an inoculum size of 10% v/v) was transferred into a secondary seed medium (50 mL) and cultured at 37° C. with shaking at 180 rpm for 7-8 hours. When an OD₅₆₂ reached 16-17, the culture was inoculated into a fermentation medium (the step (2) of Example 4) with an inoculum size of 20% (v/v), cultured at 37° C. with a shaking speed of 180 rpm for fermentation, and samples were taken every 4 hours to detect the mortality rate.

2. Detection of mortality rate

The samples were filtered through a 40 μm cell strainer to prevent cell aggregates from clogging the flow cytometer nozzle. A cell suspension concentration was adjusted to 1×10⁶-1×10⁷ CFU/mL (the concentration that was too high may cause clogging, too low may results in unstable data). Detection was performed immediately (when short-term storage was required, samples were kept at 4° C. in the dark and tested within 1 hour). The detection results are shown in Table 5. Testing results are shown in Table 5.

TABLE 5
Mortality rate (%)

| | | | | First-generation | | | Second-generation | | |
|---|---|---|---|---|---|---|---|---|---|
| | lys chassis strains | | | lys1 strains | | | lys1 strains | | |
| Time/hours | lys-1 | lys-2 | lys-3 | lys-1.1 | lys-2.1 | lys-3.1 | lys-1.2 | lys-2.2 | lys-3.2 |
| 4 | 40.2 | 45 | 38.2 | 39.8 | 39.3 | 36.4 | 36.4 | 38.4 | 36.3 |
| 8 | 30.3 | 30.2 | 35.3 | 29.4 | 26.8 | 24.3 | 24.3 | 22 | 30.9 |
| 12 | 32.2 | 30.2 | 33.6 | 32.2 | 27.9 | 27.4 | 26.5 | 28.7 | 25.5 |
| 16 | 28.6 | 33 | 36.5 | 27.4 | 32 | 30.1 | 30.1 | 32.9 | 36.7 |
| 20 | 37.3 | 41.3 | 44.6 | 37.5 | 36.5 | 43 | 36.8 | 33.9 | 40.6 |
| 24 | 39.4 | 40.3 | 45.2 | 36.6 | 37.3 | 40.2 | 35.9 | 36.2 | 40 |
| 28 | 38.8 | 42.2 | 46.5 | 32.3 | 40.8 | 44.3 | 35.5 | 39.7 | 40.5 |

Therefore, it can be concluded that after knockout of the cbrB gene and the cbrC gene, the cell mortality rate, especially at the later stage, decreases stepwise among the strains, which has a significant effect on prolonging the fermentation duration and increases the yield.

Although the present disclosure has been disclosed as above in the form of preferred embodiments, it is not intended to limit the present disclosure. Those skilled in the art can make various modifications and variations without departing from the spirit and scope of the present disclosure. Therefore, the scope of protection of the present disclosure should be defined by the claims.

What is claimed is:

1. An engineered Escherichia coli (E. coli) strain, wherein the engineered E. coli strain is an L-lysine producing strain, and the engineered E. coli strain is obtained by using E. coli as a chassis strain and silencing a cbrB gene and a cbrC gene in a genome of the E. coli.

2. The engineered E. coli strain according to claim 1, wherein an amino acid sequence of a protein encoded by the cbrB gene is set forth in SEQ ID NO:1, and an amino acid sequence of a protein encoded by the cbrC gene is set forth in SEQ ID NO:2.

3. The engineered E. coli strain according to claim 2, wherein a method of silencing comprises: replacing a gene fragment of SEQ ID NO:4 in an upstream sequence of the cbrB gene with a target gene fragment set forth in SEQ ID NO:3, and replacing a gene fragment of SEQ ID NO:5 in an upstream sequence of the cbrC gene with the target gene fragment set forth in SEQ ID NO:3.

4. A method for improving L-lysine stress tolerance of E. coli, comprising: silencing the cbrB gene and the cbrC gene in a genome of the E. coli.

5. The method according to claim 4, wherein an amino acid sequence of a protein encoded by the cbrB gene is set forth in SEQ ID NO:1, and an amino acid sequence of a protein encoded by the cbrC gene is set forth in SEQ ID NO:2.

6. The method according to claim 5, wherein a method of silencing comprises: replacing a gene fragment of SEQ ID NO:4 in an upstream sequence of the cbrB gene with a target gene fragment set forth in SEQ ID NO:3, and replacing a gene fragment of SEQ ID NO:5 in an upstream sequence of the cbrC gene with the target gene fragment set forth in SEQ ID NO:3.

7. A method for increasing a biomass at a fermentation final biomass of an L-lysine producing strain, comprising: silencing a cbrB gene and a cbrC gene in a genome of E. coli.

8. The method according to claim 7, wherein an amino acid sequence of a protein encoded by the cbrB gene is set forth in SEQ ID NO:1, and an amino acid sequence of a protein encoded by the cbrC gene is set forth in SEQ ID NO:2.

9. The method according to claim 8, wherein a method of silencing comprises: replacing a gene fragment of SEQ ID NO:4 in an upstream sequence of the cbrB gene with a target gene fragment set forth in SEQ ID NO:3, and replacing a gene fragment of SEQ ID NO:5 in an upstream sequence of the cbrC gene with the target gene fragment set forth in SEQ ID NO:3.

10. A preparation method of L-lysine, wherein the L-lysine is prepared by fermentation using the engineered E. coli strain in claim 1.

11. The preparation method according to claim 10, wherein a fermentation temperature is 30-37° C., and a fermentation duration is 30-42 hours.

12. The preparation method according to claim 11, comprising: inoculating a seed culture of the engineered E. coli strain into a fermentation medium containing glucose, followed by fermentation.

13. The preparation method according to claim 12, wherein the seed culture is added at a volume ratio of 15-20%.

14. The preparation method according to claim 13, wherein a preparation method of the seed culture comprises: streaking the engineered E. coli strain onto an agar slant medium, culturing at a temperature of 33-37° C. for 12-16 hours, and inoculating into a primary seed medium at an inoculum size of 5-15% (v/v); and culturing at a temperature of 33-37° C. until OD₅₆₂ reaches 4.0-5.0 to obtain a primary seed culture, and inoculating the obtained primary seed culture into a secondary seed medium at an inoculum size of 5-15% (v/v), and culturing at a temperature of 33-37° C. until OD₅₆₂ reaches 15.0-18.0.


SEQUENCE LISTING (37 sequences)

SEQ ID NO: 1 - protein, 155 aa, synthetic construct
MSVSRRVIHHGLYFAVLGPLIGVLFLVLYIFFAKEPLVLWVIIHPIFLLLSITTGAIPALLTGVMVACLPEKIGSQKRYRCLAGGIGGVVITEIYCAVIVHIKGMASSELFENILSGDSLVVRIIPALLAGVVMSRIITRLPGLDISCPETDSLS

SEQ ID NO: 2 - protein, 195 aa, synthetic construct
MTQNIRPLPQFKYHPKPLETGAFEQDKTVECDCCEQQTSVYYSGPFYCVDEVEHLCPWCIADGSAAEKFAGSFQDDASIEGVEFEYDEEDEFAGIKNTYPDEMLKELVERTPGYHGWQQEFWLAHCGDFCVFIGYVGWNDIKDRLDEFANLEEDCENFGIRNSDLAKCLQKGGHCQGYLFRCLHCGKLRLWGDFS

SEQ ID NO: 3 - DNA, 1784 bp, synthetic construct
  source 1..1784: mol_type = other DNA
ttgggtgttgtttgaagtataagttgacatatctgtactaaaagatggcataaattggaagtgtaaggtggcatagtctagtatttaaccaaatggttaaatggttgactcaccgggaattcgagataaattgcactgaaatctagaggtcgaaattcacctcgaaagcaagctgataaaccgatacaattaaaggctccttttggagcctttttttttggagattttcaacgtgaaaaaattattattcgcaattccaagctaattcacctcgaaagcaagctgataaaccgatacaattaaaggctccttttggagcctttttttttggagattttcaacgtgaaaaaattattattcgcaattccaagctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatgggctcgatcccctcggatcgatccccatgtaatgaataaaaagcagtaattaatacatctgtttcatttgaagcgcgaaagctaaagttttcgcatttatcggatccggctgctaatcgagttaattaaactagtgagctcggtacccggggatcctctagaggtcgaaattcacctcgaaagcaagctgataaaccgatacaattaaaggctccttttggagcctttttttttggagattttcaacgtgaaaaaattattattcgcaattccaagctaattcacctcgaaagcaagctgataaaccgatacaattaaaggctccttttggagcctttttttttggagattttcaacgtgaaaaaattattattcgcaattccaagctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatgggctcgatcccctcggatccaattggtcttaattttccttaattatttttaaagttagactgatttagacttggaaaagcttaatgattggagagctaaattgactaatagtattcagtcaagtttaattagttttaagcgatccatgcataactatttctgtacaatgctatattttcaccaattaataatttttaatcaagcctacattatgaaatatactatacccatttgaactcttctatttgtaccattgtcggtagcaaaaacttatggggttttgaacgtcacttaaattgtaagcatttgcgatggaggcgcgtttagagtcaaccttgattctgatatgctccgaatttttggtaagaatataagtgtgagagtagctaatgtggatacgcctgagttaagggaaaaatgtgaaaatgaaataactcgttatcatgcaaagtgactaaggttataatcttccgtttatggcacatagcagccaactaaacttgacagtatttttatgtggttggctttataaaaccagcatttggtaacattatgccaatttttacttcaatattatgccaacatacactacactaacggagctgtagcacaataagctcgtttgtacttatgccaacttatacttcaaacaacattgg

SEQ ID NO: 4 - DNA, 32 bp, synthetic construct
  source 1..32: mol_type = other DNA
ctgtctgccggaaaagatcgggtcacagaaac

SEQ ID NO: 5 - DNA, 32 bp, synthetic construct
  source 1..32: mol_type = other DNA
aaagtttgcaggtagttttcaggatgatgcca

SEQ ID NO: 6 - DNA, 6061 bp, synthetic construct
  source 1..6061: mol_type = other DNA
ctgtctgccggaaaagatcgggtcacagaaacgtgaactgccgagtaggcagctgaagttggatccgaattcgagctcgggagaacttatcggtggtggtgtcacgctgtctgccggaaaagatcgggtcacagaaacgtgaactgccgagtaggcagctgaagttggatccgaattcgagctcgggaagaaggagatatacatatgcactttctggtgcagaccaagagctacccggacgaggcgctggaaagctatctgctgcgtctggcgcgtgataacagctacaacggttatagcgagctggcggacatcctgtggcagtggctggcggaacaagataacgagctggaaggtgcgctgccgctggcgctgagcaaggtggacgtttaccacgcgcgtcaggcgagcagcttccgtatccgtgcgctgaaactggtggcgcaactggcggacgttaacgcgggtgatattctggcgctggcgtggcgtcgtagcaacttcaagtttggcaacctggcggcggtgagccgtaacgagctggcgatcccgctggaactgctgcgtaccgataacatcccggtttgcattaaatgcctgagcgagagcagccacattccgttttactggcacctgaagccgtataaagcgtgccacaagcacaaaagccagctgatcacccgttgcaaggagtgctacgacctgattgattatcgtgcgagcgaggcgtttctggaatgcgtttgcggttgcaaaatcaccaacagcgaacaactgaacgacgcggatttcaagatcgcgattgcgctggcgagcagcaacagccagaaaatcgtgggcctgattagctggttcgcgaaggtgaaacaactggacgttagcgacgcggatttcaactgcgcgtttgttgattacttcaacacctggccggagagcctgaccaccgaactggacctgctgaccaacaacgcgcgtctgaagcagctgaacccgtttaacaagaccaaattcagcagcgtgtacggtgacctgatccgtgatggccaaattgcggcgaccagcaaccgtaagaacaaagttatcgacgagatcattagctattttgtggaactggttgatagcaacccgaaggcgaaacacccgaacattggtgacctgctgctgtgcaccttcgatgcggcggtgctgctgaacaccaccaccgagcaggtttaccgtctgcaccaagaagcgtttctgaactgcgcgtatagccagaagaaacacgaacaactgcgtgcggatagccacgtgttctatctgcgtcaggttatcgagctgcagcaagcgtttgcggcggaaaaaccgctgaccaagaaacaattcattgcgccgtggtaacttatgaacctgcaggatgcgctggcgattgagccgctgaaggaaaaaaccaccgcgctgcgtaagctgttcgtgccgtacaccagccacgttgaggtggatggttttgaggaactggcgctgaccgtgctgatcaacctggtttataagcgtagcgaaattgacgatctgaccagcgcgcgtaccgcgaaaagcgtgctgcgtgacgaggttctgctgagcaagtgcatcaacgaagtgaaatggttccacacccacaacctgaagtacccggacatccgtgttagccaccaacgtctgattagcgaggtggttagcgaagatatcgcgggtatttgcagccgtagcctgccgctgagctttggctggagccacaacagcgcggagatcaaccacgcgaaactgttcctgaccagctttaactggcagggtgaagtgacctgcctggcgcgtctgctgattaacgaggaaccggtttggatcaacctgattcgtgcgtacggtttcaccaagaaagcggttctggagatcagcggcaagattaaacagcaactgccggtggcggagttcccgctggaagttagcagctttagcccgcagctgcaaatgccgtttcagcaaagctatctggtggttaccccggtggttagccacgcgatgctggcgaagatccagcaactgaccaccgaccgtaaactgaacttcgcgctggttgagcacagccgtccggcgaacgttggtgatctggcgagcagcgtgggtggcaacattcgtgttctgcgttactttccgaagacctatagcaaagcggtgaaccgtagcaaagttgcgaacaacgatatcgaaaaggcgttcaaaattcgtgcgctgctgagcagccagtttcagcaagcgctgctggtgctggttggcatcaagcagttcaacaccctgcgtcaaaaacgtctggcgcgtgtggcggcgatccgtcaagtgcgtgttagcctgcaactgtggctggacaacattctggaggcgaagaacaacgcgcagaaccaagtgtacccggaatgggttcgtcactatctggatcaaagcatcaccaactgcattagccagttcagcaacgttctgaacgaaagcctgggtaacctgagcaagctgaaacgttttgcgtaccacccgaacctgatgggcctgttcaaagcgcaactgaactatgtgtttacccactgcgcggcggagcaggaaatcctgaacgacgagcaaattgtgtacgttcactgccaggacatgcgtgttttcgatgcggaagcgatggcgaacccgtatatccagggtatgccgagcctgaccgcgctgaacggcctggcgcacaacttcgagcgtaagctgaaaaactttattgatccgagcatcaagtgcattggtagcgcgatctacattgagaactatcaactgcacaccggcaaaccgctgccggaaccgagcaagctgaaacaggtggcgggtcgtagccacgttatccgtagcggcatcattgacaagccgaaatgcgacattaccctggatctggtgttccgtctgtttgttccgaacaccgaactgctggataagctgaacagccaactgattaagccggcgctgccgagcagctttgcgggtggcaccatgcacccgccgagcctgtaccagaacattgactggtgccacgtgcacaccaagccgagcgagctgtttaagaaactgaaggcgaaaagcagcaacggtagctggctgtatccgagcaagaaagtggttaaaagcttcgaacagctgatcgacgcgctgaacagcaactttaacctgcgtccggcggcgattggcctggcggcgctggaggaaccggtgaagcgtgatgcggcgctgcacgagtaccactgctatgcggaaccggttatcggtctgctggagtgcgtgagcaacaccagcgttaagtacgcgggcgcgaaacaattctttcacgacgcgttctgggtgatggatgttcagaaggaaagcatgctgatgaagaaaagcaaatttgagtatgaataatgcagctgccgcgtcacctgagctacacccgtagcctgagcccgagcaaggcggtgttcttttataaaaccccggagagcgacttcgaaccgctgcagatcgagcaaaacaaactggtgggtcagaagagcggttttggcgatgcgtaccagaagcaaaacgttgcgaaaaacctggcgccgcaggacctggcgtttggtaacccgcaaaccattgatgtgtgctatgttccgccgaccgtgaacgaactgttctgccgttttagcctgcgtgttgaggcgaactgcatcgaaccgcacgtgtgcgacgatccgaaggttatttactggctgaaacgtttctttgaaacctataagaaacacaacggtctgaacgaagtggcgacccgttacgcgaagaacatcctgatgggcaactggctgtggcgtaaccgtcagagcccgaacgttgacatcgagattctgaccgaacacgcggcgccgattgtggttgagggtgcgcagaagctgaaatggcaaggcaactggcagaacaaccaaaccgcgctgctgaccctgagcgagagcatccaggaaggtctgagcaacccgcaaaactactgctatctggatatcaccgcgaagattaaaaacgcgttcagccaggaagtgcacccgagccaaaagtttgtggacaacgttgaacagggtatgagcagcaaacagctggcgtatacccaagtgggcgataagaaagcggcgagcctgaacagccagaaggttggcgcggcgatccaaaccattgacgattggtacgaggaaggttataaaccgctgcgtacccatgagtatggtgcggacaagcaaatcctggtggcgcaccgtaccccgaaaagccacagcgatttttatagcctgctgccgcgtatcgcgctgcacattaagcacatggaaaaacacggtctggagcagagcgaacaaagcaacagcatccacttcattgcggcggttctgattaagggtggcctgtttcagcgtagcaaaggatgaagcgttactatttcaccatcacctacctgccgcaaagctgcgatgtgagcctgctggcgggtcgttgcatcggcattctgcacggtttcatgagcagccgtgagatcagcaacattggcgtgtgctttccgaaatggaacgagcagaccatcggtaacgaactggcgtttgttagcaccaacaagaaacaactgaccaacctgagccagcaaagctatttcgagatgatggcgcacgacaagctgtttggcctgagcaaaattctggaagtgccggttaaccagagcgaagtgatgttcgttcgtaaccaaagcgtggcgaaggcgtttgttggtgaaaagcaacgtcgtctgaaacgtgcgaagaaacgtgcggaggcgcgtggcgaagtgtacaacccggagtataagttcgaagcgaaagatatcggtcactttcacagcattccggtgagcagcaagggtaacggccagagctacgttctgcacatccaaaagaacgagaacgcggaaagcattaaaaaccagttcaacaactatggctttgcgaccaaccaaattttcctgggcaccgtgccgagcctgaacaccctgctgtaaaaggagatataccatgtaccgtcgtaagctgaaatatagccgtgttaagaacctgcacaaatttgcgagccagaagaacaaaagcacctgcctggtggagagcagcctggaattcgacgcgtgcttccactttgagttcagcccgccgatcgcggcgtttgaagcgcaaccgctgggttacgagtatgaattcgataaccgtatttgccgttacaccccggactttctgctgacccacaccgatggcacccagaagttcatcgaggttaagccgcaaagcaaaattgcggacgaggattttcgtgcgcgtttcatcgaaaagcaggcgattgcgaaacaagacggtcgtgatctgatcctggtgaccgacaagcagattcgtgtttacccgaccctgaacaacctgaaactgctgcaccgttatagcggctttcagagcctgaccgagctgcaagcgagcgtgctggaactggttaagcagtacggtagcatcaaagtgggccaactgattcgttatctgaaagttaccgcgggtgaactgctggcgaccgtgctgcgtctgctgagcctgggccaactgttcgcggatctgaccaccaacgagatcagcattgaaaccgcgatctggagcaacaatgtttaataacgacctgttcgacgatgagtttaaccagccgctgccgaaggcggaaaccaaactgccgcagaactataccaaggatctgcaagcgctgccggagaagatcaaaaccaccaccttcgcgaagctgaaatacattcaatggctggaggcgaacatccagggtggctggacccaaaagaacctggaaccgctgctgaaactgatgccggacgttgagggtgaaaagaaaccgagctggcgtaccgcggcgcgttggtatagcgcgtacaccaacgcggataagaacattatggcgctgatcccgagccaccagaagaaaggcaaccgtgaacgtgacaccaccaccgataagttct

SEQ ID NO: 7 - DNA, 5532 bp, synthetic construct
  source 1..5532: mol_type = other DNA
gtttctgtgacccgatcttttccggcagacagatttccagctgcctactcggcagttcacccatggtatatctccttttaaagtcgtgacaccaccaccgataagttctcgtgacaccaccaccgataagttctttgagaaagcgctggaacgttacctggtgaaggagaaaccgagcgttgcgagcgcgtataagttctacaaagacctggtgatcattgaaaacgacagcgtggttgatagcgttctgaaaccgctgacctataaggcgtttaaaaaccgtattgacaacctgccgcagtatgaggttatgatcgcgcgttacggcaagcgtctggcggatattgcgtacaacaaggtggaaggccacaaacgtccgattcgtgtgctggagaaagttgaaatcgaccacaccccgctggatctgattctgctggacgatgagctgcacatcccgctgggtcgtccgaccctgaccatgctggttgacgtttatagccactgcatcgtgggctactatttcagctttagcgagccgagctacgatgcggttcgtcgtgcgatgctgaacgcgatgaagccgaaaagcgaagtggcgaaactgtacccggacaccattaacgagtggaagtgcgcgggtaaaatcgaaaccctggtggttgataacggcgcggagttctggagcaacagcctggaactggcgtgcgaggaaatcggtattaacacccagtataacccggtggcgaagccgtggctgaaaccgttcgttgagcgtatgtttggcaccatcaacaccgaactgctggacccggttccgggcaagaccttcagcaacatcctgcaaaaacacgaatacaacccgaagaaagacgcgattatgcgtttcaccacctttatgcagctgtttcacaagtgggtggttgatgtgtatcaccaagacgcggatagccgtttcaaatacattccgagccagctgtgggaccaaggctttaacaccctgccgccgaccatgctgagcgatgcggatctgcagcaactggatgtggttctgagcatcagcaaccaccgtgtgctgcgtaagggtggcattcgtctggagaacctgagctatgacagcaccgaactggcgaactaccgtaagcagttcagccacaaagtgagccaagaggttctgatcaaactgaacccggacgatattagctacatctatgtgtacctggacaagctggaacactatattaaagttccgtgcatcgatccgaacggttacacccagaacctgagcctgaaccaacacaagatcaacattcgtatccaccgtgactttattagcggtagcatcgataacgttggcctggcgaaggcgcgtatgttcattcacaacaaaatccagaacgagtttgaggaactgaagaacgcgccgaaacacagcaaggtgaaaggtggcaaggcgctggcgaaacaccagaacattagcagcgacagccaaaagagcatcacccacagcaaaccggtggaggcgaagaaagttaccccgaaagaacaaccgaccgatagctgggacgatttcatcagcgacctggatggtttttaattatgctgaccgacaagcagaaagaaaagctgaacgagttccgtgatgtttttattgaatacccgatcattaccaccatcttcaacgactttgatcgtctgcgtctgggtaaaggcctgaccggcgagaagccgtgcatgctgctgaacggtgacaccggcaccggtaaaaccgcgctgattaaacagtataaggaacgtcacctgccgcaattcatcaacggtgttatgaaccacccggtgctggttagccgtattccgagcaacccgaccctggaaagcaccctggcggagctgctgaaagacctgggtcaagtgggcagcaccgagcgtaagctgcgtattaacggcacccgtctgaccaccagcctgatcaaatgcctgaagacctgcggcaccgaactgatcattatcgatgagtttcaggaactgattgagcacaaccaaggcaagaaacgtcgtgaaattgcgaaccgtctgaaatacatcaacgacgaggcgggtgttagcattgtgctggttggcatgccgtgggcggaaaagatcgcggatgagccgcagtggagcagccgtctgctgatccgtcgtcaactgccgtatttcaaactgagcgagaacccgaagcactttgtgcagctgattatcggtctggcgaaccgtatgccgttcgcggaaaaaccgaacctgagcgagcaagcgaccgttttcaccctgtttagcctgagcaaaggctgcttccgtaccctgaagtactttctggacgatgcggtgctgtatgcgctgatggacaacgcgaagaccctgaccaccaaacacctggtgaaggcgttcgaagttctgtttccggatgtgccgaacctgtttaccctgccggttgcggagatcaccgcgagcgaggtggaacgttacagcctgtataagccggaaagcagccaggacgaggacccgttcattgcgaccaaatttaccgatcgtatgccgatcagccaactgctgcgtaagtaactcgagccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctgatgaatcccctaatgatttttatcaaaatcattaaggttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaaaaggagatataccatgggtgaactgccgagtaggcagctggaaatctgtctgccggaaaagatcgggtcacagaaac

SEQ ID NO: 8 - DNA, 6061 bp, synthetic construct
  source 1..6061: mol_type = other DNA
aaagtttgcaggtagttttcaggatgatgccagtgaactgccgagtaggcagctgaagttggatccgaattcgagctcgggagaacttatcggtggtggtgtcacgaaagtttgcaggtagttttcaggatgatgccagtgaactgccgagtaggcagctgaagttggatccgaattcgagctcgggaagaaggagatatacatatgcactttctggtgcagaccaagagctacccggacgaggcgctggaaagctatctgctgcgtctggcgcgtgataacagctacaacggttatagcgagctggcggacatcctgtggcagtggctggcggaacaagataacgagctggaaggtgcgctgccgctggcgctgagcaaggtggacgtttaccacgcgcgtcaggcgagcagcttccgtatccgtgcgctgaaactggtggcgcaactggcggacgttaacgcgggtgatattctggcgctggcgtggcgtcgtagcaacttcaagtttggcaacctggcggcggtgagccgtaacgagctggcgatcccgctggaactgctgcgtaccgataacatcccggtttgcattaaatgcctgagcgagagcagccacattccgttttactggcacctgaagccgtataaagcgtgccacaagcacaaaagccagctgatcacccgttgcaaggagtgctacgacctgattgattatcgtgcgagcgaggcgtttctggaatgcgtttgcggttgcaaaatcaccaacagcgaacaactgaacgacgcggatttcaagatcgcgattgcgctggcgagcagcaacagccagaaaatcgtgggcctgattagctggttcgcgaaggtgaaacaactggacgttagcgacgcggatttcaactgcgcgtttgttgattacttcaacacctggccggagagcctgaccaccgaactggacctgctgaccaacaacgcgcgtctgaagcagctgaacccgtttaacaagaccaaattcagcagcgtgtacggtgacctgatccgtgatggccaaattgcggcgaccagcaaccgtaagaacaaagttatcgacgagatcattagctattttgtggaactggttgatagcaacccgaaggcgaaacacccgaacattggtgacctgctgctgtgcaccttcgatgcggcggtgctgctgaacaccaccaccgagcaggtttaccgtctgcaccaagaagcgtttctgaactgcgcgtatagccagaagaaacacgaacaactgcgtgcggatagccacgtgttctatctgcgtcaggttatcgagctgcagcaagcgtttgcggcggaaaaaccgctgaccaagaaacaattcattgcgccgtggtaacttatgaacctgcaggatgcgctggcgattgagccgctgaaggaaaaaaccaccgcgctgcgtaagctgttcgtgccgtacaccagccacgttgaggtggatggttttgaggaactggcgctgaccgtgctgatcaacctggtttataagcgtagcgaaattgacgatctgaccagcgcgcgtaccgcgaaaagcgtgctgcgtgacgaggttctgctgagcaagtgcatcaacgaagtgaaatggttccacacccacaacctgaagtacccggacatccgtgttagccaccaacgtctgattagcgaggtggttagcgaagatatcgcgggtatttgcagccgtagcctgccgctgagctttggctggagccacaacagcgcggagatcaaccacgcgaaactgttcctgaccagctttaactggcagggtgaagtgacctgcctggcgcgtctgctgattaacgaggaaccggtttggatcaacctgattcgtgcgtacggtttcaccaagaaagcggttctggagatcagcggcaagattaaacagcaactgccggtggcggagttcccgctggaagttagcagctttagcccgcagctgcaaatgccgtttcagcaaagctatctggtggttaccccggtggttagccacgcgatgctggcgaagatccagcaactgaccaccgaccgtaaactgaacttcgcgctggttgagcacagccgtccggcgaacgttggtgatctggcgagcagcgtgggtggcaacattcgtgttctgcgttactttccgaagacctatagcaaagcggtgaaccgtagcaaagttgcgaacaacgatatcgaaaaggcgttcaaaattcgtgcgctgctgagcagccagtttcagcaagcgctgctggtgctggttggcatcaagcagttcaacaccctgcgtcaaaaacgtctggcgcgtgtggcggcgatccgtcaagtgcgtgttagcctgcaactgtggctggacaacattctggaggcgaagaacaacgcgcagaaccaagtgtacccggaatgggttcgtcactatctggatcaaagcatcaccaactgcattagccagttcagcaacgttctgaacgaaagcctgggtaacctgagcaagctgaaacgttttgcgtaccacccgaacctgatgggcctgttcaaagcgcaactgaactatgtgtttacccactgcgcggcggagcaggaaatcctgaacgacgagcaaattgtgtacgttcactgccaggacatgcgtgttttcgatgcggaagcgatggcgaacccgtatatccagggtatgccgagcctgaccgcgctgaacggcctggcgcacaacttcgagcgtaagctgaaaaactttattgatccgagcatcaagtgcattggtagcgcgatctacattgagaactatcaactgcacaccggcaaaccgctgccggaaccgagcaagctgaaacaggtggcgggtcgtagccacgttatccgtagcggcatcattgacaagccgaaatgcgacattaccctggatctggtgttccgtctgtttgttccgaacaccgaactgctggataagctgaacagccaactgattaagccggcgctgccgagcagctttgcgggtggcaccatgcacccgccgagcctgtaccagaacattgactggtgccacgtgcacaccaagccgagcgagctgtttaagaaactgaaggcgaaaagcagcaacggtagctggctgtatccgagcaagaaagtggttaaaagcttcgaacagctgatcgacgcgctgaacagcaactttaacctgcgtccggcggcgattggcctggcggcgctggaggaaccggtgaagcgtgatgcggcgctgcacgagtaccactgctatgcggaaccggttatcggtctgctggagtgcgtgagcaacaccagcgttaagtacgcgggcgcgaaacaattctttcacgacgcgttctgggtgatggatgttcagaaggaaagcatgctgatgaagaaaagcaaatttgagtatgaataatgcagctgccgcgtcacctgagctacacccgtagcctgagcccgagcaaggcggtgttcttttataaaaccccggagagcgacttcgaaccgctgcagatcgagcaaaacaaactggtgggtcagaagagcggttttggcgatgcgtaccagaagcaaaacgttgcgaaaaacctggcgccgcaggacctggcgtttggtaacccgcaaaccattgatgtgtgctatgttccgccgaccgtgaacgaactgttctgccgttttagcctgcgtgttgaggcgaactgcatcgaaccgcacgtgtgcgacgatccgaaggttatttactggctgaaacgtttctttgaaacctataagaaacacaacggtctgaacgaagtggcgacccgttacgcgaagaacatcctgatgggcaactggctgtggcgtaaccgtcagagcccgaacgttgacatcgagattctgaccgaacacgcggcgccgattgtggttgagggtgcgcagaagctgaaatggcaaggcaactggcagaacaaccaaaccgcgctgctgaccctgagcgagagcatccaggaaggtctgagcaacccgcaaaactactgctatctggatatcaccgcgaagattaaaaacgcgttcagccaggaagtgcacccgagccaaaagtttgtggacaacgttgaacagggtatgagcagcaaacagctggcgtatacccaagtgggcgataagaaagcggcgagcctgaacagccagaaggttggcgcggcgatccaaaccattgacgattggtacgaggaaggttataaaccgctgcgtacccatgagtatggtgcggacaagcaaatcctggtggcgcaccgtaccccgaaaagccacagcgatttttatagcctgctgccgcgtatcgcgctgcacattaagcacatggaaaaacacggtctggagcagagcgaacaaagcaacagcatccacttcattgcggcggttctgattaagggtggcctgtttcagcgtagcaaaggatgaagcgttactatttcaccatcacctacctgccgcaaagctgcgatgtgagcctgctggcgggtcgttgcatcggcattctgcacggtttcatgagcagccgtgagatcagcaacattggcgtgtgctttccgaaatggaacgagcagaccatcggtaacgaactggcgtttgttagcaccaacaagaaacaactgaccaacctgagccagcaaagctatttcgagatgatggcgcacgacaagctgtttggcctgagcaaaattctggaagtgccggttaaccagagcgaagtgatgttcgttcgtaaccaaagcgtggcgaaggcgtttgttggtgaaaagcaacgtcgtctgaaacgtgcgaagaaacgtgcggaggcgcgtggcgaagtgtacaacccggagtataagttcgaagcgaaagatatcggtcactttcacagcattccggtgagcagcaagggtaacggccagagctacgttctgcacatccaaaagaacgagaacgcggaaagcattaaaaaccagttcaacaactatggctttgcgaccaaccaaattttcctgggcaccgtgccgagcctgaacaccctgctgtaaaaggagatataccatgtaccgtcgtaagctgaaatatagccgtgttaagaacctgcacaaatttgcgagccagaagaacaaaagcacctgcctggtggagagcagcctggaattcgacgcgtgcttccactttgagttcagcccgccgatcgcggcgtttgaagcgcaaccgctgggttacgagtatgaattcgataaccgtatttgccgttacaccccggactttctgctgacccacaccgatggcacccagaagttcatcgaggttaagccgcaaagcaaaattgcggacgaggattttcgtgcgcgtttcatcgaaaagcaggcgattgcgaaacaagacggtcgtgatctgatcctggtgaccgacaagcagattcgtgtttacccgaccctgaacaacctgaaactgctgcaccgttatagcggctttcagagcctgaccgagctgcaagcgagcgtgctggaactggttaagcagtacggtagcatcaaagtgggccaactgattcgttatctgaaagttaccgcgggtgaactgctggcgaccgtgctgcgtctgctgagcctgggccaactgttcgcggatctgaccaccaacgagatcagcattgaaaccgcgatctggagcaacaatgtttaataacgacctgttcgacgatgagtttaaccagccgctgccgaaggcggaaaccaaactgccgcagaactataccaaggatctgcaagcgctgccggagaagatcaaaaccaccaccttcgcgaagctgaaatacattcaatggctggaggcgaacatccagggtggctggacccaaaagaacctggaaccgctgctgaaactgatgccggacgttgagggtgaaaagaaaccgagctggcgtaccgcggcgcgttggtatagcgcgtacaccaacgcggataagaacattatggcgctgatcccgagccaccagaagaaaggcaaccgtgaacgtgacaccaccaccgataagttct

SEQ ID NO: 9 - DNA, 5532 bp, synthetic construct
  source 1..5532: mol_type = other DNA
tggcatcatcctgaaaactacctgcaaactttatttccagctgcctactcggcagttcacccatggtatatctccttttaaagtcgtgacaccaccaccgataagttctcgtgacaccaccaccgataagttctttgagaaagcgctggaacgttacctggtgaaggagaaaccgagcgttgcgagcgcgtataagttctacaaagacctggtgatcattgaaaacgacagcgtggttgatagcgttctgaaaccgctgacctataaggcgtttaaaaaccgtattgacaacctgccgcagtatgaggttatgatcgcgcgttacggcaagcgtctggcggatattgcgtacaacaaggtggaaggccacaaacgtccgattcgtgtgctggagaaagttgaaatcgaccacaccccgctggatctgattctgctggacgatgagctgcacatcccgctgggtcgtccgaccctgaccatgctggttgacgtttatagccactgcatcgtgggctactatttcagctttagcgagccgagctacgatgcggttcgtcgtgcgatgctgaacgcgatgaagccgaaaagcgaagtggcgaaactgtacccggacaccattaacgagtggaagtgcgcgggtaaaatcgaaaccctggtggttgataacggcgcggagttctggagcaacagcctggaactggcgtgcgaggaaatcggtattaacacccagtataacccggtggcgaagccgtggctgaaaccgttcgttgagcgtatgtttggcaccatcaacaccgaactgctggacccggttccgggcaagaccttcagcaacatcctgcaaaaacacgaatacaacccgaagaaagacgcgattatgcgtttcaccacctttatgcagctgtttcacaagtgggtggttgatgtgtatcaccaagacgcggatagccgtttcaaatacattccgagccagctgtgggaccaaggctttaacaccctgccgccgaccatgctgagcgatgcggatctgcagcaactggatgtggttctgagcatcagcaaccaccgtgtgctgcgtaagggtggcattcgtctggagaacctgagctatgacagcaccgaactggcgaactaccgtaagcagttcagccacaaagtgagccaagaggttctgatcaaactgaacccggacgatattagctacatctatgtgtacctggacaagctggaacactatattaaagttccgtgcatcgatccgaacggttacacccagaacctgagcctgaaccaacacaagatcaacattcgtatccaccgtgactttattagcggtagcatcgataacgttggcctggcgaaggcgcgtatgttcattcacaacaaaatccagaacgagtttgaggaactgaagaacgcgccgaaacacagcaaggtgaaaggtggcaaggcgctggcgaaacaccagaacattagcagcgacagccaaaagagcatcacccacagcaaaccggtggaggcgaagaaagttaccccgaaagaacaaccgaccgatagctgggacgatttcatcagcgacctggatggtttttaattatgctgaccgacaagcagaaagaaaagctgaacgagttccgtgatgtttttattgaatacccgatcattaccaccatcttcaacgactttgatcgtctgcgtctgggtaaaggcctgaccggcgagaagccgtgcatgctgctgaacggtgacaccggcaccggtaaaaccgcgctgattaaacagtataaggaacgtcacctgccgcaattcatcaacggtgttatgaaccacccggtgctggttagccgtattccgagcaacccgaccctggaaagcaccctggcggagctgctgaaagacctgggtcaagtgggcagcaccgagcgtaagctgcgtattaacggcacccgtctgaccaccagcctgatcaaatgcctgaagacctgcggcaccgaactgatcattatcgatgagtttcaggaactgattgagcacaaccaaggcaagaaacgtcgtgaaattgcgaaccgtctgaaatacatcaacgacgaggcgggtgttagcattgtgctggttggcatgccgtgggcggaaaagatcgcggatgagccgcagtggagcagccgtctgctgatccgtcgtcaactgccgtatttcaaactgagcgagaacccgaagcactttgtgcagctgattatcggtctggcgaaccgtatgccgttcgcggaaaaaccgaacctgagcgagcaagcgaccgttttcaccctgtttagcctgagcaaaggctgcttccgtaccctgaagtactttctggacgatgcggtgctgtatgcgctgatggacaacgcgaagaccctgaccaccaaacacctggtgaaggcgttcgaagttctgtttccggatgtgccgaacctgtttaccctgccggttgcggagatcaccgcgagcgaggtggaacgttacagcctgtataagccggaaagcagccaggacgaggacccgttcattgcgaccaaatttaccgatcgtatgccgatcagccaactgctgcgtaagtaactcgagccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctgatgaatcccctaatgatttttatcaaaatcattaaggttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaaaaggagatataccatgggtgaactgccgagtaggcagctggaaataaagtttgcaggtagttttcaggatgatgcca

SEQ ID NO: 10 - DNA, 32 bp, synthetic construct
  source 1..32: mol_type = other DNA
tggatgctttgcgtgcaaaggaacctgaactc

SEQ ID NO: 11 - DNA, 550 bp, synthetic construct
  source 1..550: mol_type = other DNA
ttcttcacactcccttcacttaccccgcttaaattggcgctcaaaggtaagtaaagggagtttgatatgtctgtttcacgtcgggtaatacatcacggactttattttgcagttttaggaccgttaattggtgttctgtttcttgtcctctacatattcttcgcaaaagaaccgctggttctttgggtgataatacatccaatttttctcttattgtcgataactacgggagctattcctgcgttgttaaccggtgtaatggttgcgttatcgttgtctggctggtggcataggtggcgtcgttatcaccgagatctattgtgcagttattgtacatattaagggcatggcttcctcggagttgtttgaaaacattctttctggtgacagtctcgttgtccgcatcattcctgcattgctggcaggtgtggtgatgagcagaatcattacccgtctacccggattggatatttcatgtcctgaaacagactctttaagttaagcgggatactttatctttgggctactcaaaagcagacaggatgtttct

SEQ ID NO: 12 - DNA, 629 bp, synthetic construct
  source 1..629: mol_type = other DNA
gcgggatactttatctttgggctactcaaaagcagacaggatgtttctatgactcaaaatatcaggccgttaccccaattcaaatatcatcccaagccactggaaacaggcgcatttgaacaggataaaaccgtagagtgcgattgctgtgaacaacagacgtcagtttattactcgggtcccttttattgcgttgatgaagttgaacatctctgtccgtggtgtattgcggacggttctgctgctgagcatagaaggtgttgaatttgagtatgatgaagaggacgaatttgccggtattaagaacacatatcctgatgaaatgctgaaagagttggttgaacgcacgccaggttatcatggatggcagcaggaattctggctcgcgcattgtggcgatttctgtgtttttatcggctatgtgggctggaatgatataaaagatcgcctcgatgaatttgccaaccttgaagaagattgtgagaatttcggtattagaaattctgatctagctaaatgcctgcaaaagggtggtcattgtcagggttatctcttccgctgtctccactgcggcaagctgagactgtggggtgatttttcgtagttatttaaataatgagaacaggccg

SEQ ID NO: 13 - DNA, 2214 bp, synthetic construct
  source 1..2214: mol_type = other DNA
atgtctgtttcacgtcgggtaatacatcacggactttattttgcagttttaggaccgttaattggtgttctgtttcttgtcctctacatattcttcgcaaaagaaccgctggttctttgggtgataatacatccaatttttctcttattgtcgataactacgggagctattcctgcgttgttaaccggtgtaatggttgcttgggtgttgtttgaagtataagttgacatatctgtactaaaagatggcataaattggaagtgtaaggtggcatagtctagtatttaaccaaatggttaaatggttgactcaccgggaattcgagataaattgcactgaaatctagaggtcgaaattcacctcgaaagcaagctgataaaccgatacaattaaaggctccttttggagccttttttttggagattttcaacgtgaaaaaattattattcgcaattccaagctaattcacctcgaaagcaagctgataaaccgatacaattaaaggctccttttggagccttttttttggagattttcaacgtgaaaaaattattattcgcaattccaagctctgcctcgcgcgtttcggtgatgacggtgaaaacctctgacacatgcagctcccggagacggtcagcttgtctgtaagcggatgccgggagcagacaagcccgtcagggcgcgtcagcgggtttggcgggtgtcggggcgcagccatgacccagtcacgtagcgatagcggagtgtatgggctcgatcccctcggatcgatccccatgtaatgaataaaagcagtaattaatacatctgtttcatttgaagcgcgaaagctaaagttttcgcatttatcggatccggctgctaatcgagttaattaaactagtgagctcggtacccggggatcctctagaggtcgaaattcacctcgaaagcaagctgataaaccgatacaattaaaggctccttttggagcctttttttttggagattttcaacgtgaaaaaattattattcgcaattccaagctaattcacctcgaaagcaagctgataaaccgatacaattaaaggctccttttggagcctttttttttggagattttcaacgtgaaaaaattattattcgcaattccaagctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatgggctcgatcccctcggatccaattggtcttaattttccttaattatttttaaagttagactgatttagacttggaaaagcttaatgattggagagctaaattgactaatagtattcagtcaagtttaattagttttaagcgatccatgcataactatttctgtacaatgctatattttcaccaattaataatttttaatcaagcctacattatgaaatatactatacccatttgaactcttctatttgtaccattgtcggtagcaaaaacttatggggttttgaacgtcacttaaattgtaagcatttgcgatggaggcgcgtttagagtcaaccttgattctgatatgctccgaatttttggtaagaatataagtgtgagagtagctaatgtggatacgcctgagttaagggaaaaatgtgaaaatgaaataactcgttatcatgcaaagtgactaaggttataatcttccgtttatggcacatagcagccaactaaacttgacagtatttttatgtggttggctttataaaaccagcatttggtaacattatgccaatttttacttcaatattatgccaacatacactacactaacggagctgtagcacaataagctcgtttgtacttatgccaacttatacttcaaacaacattgggttatcgttgtctggctggtggcataggtggcgtcgttatcaccgagatctattgtgcagttattgtacatattaagggcatggcttcctcggagttgtttgaaaacattctttctggtgacagtctcgttgtccgcatcattcctgcattgctggcaggtgtggtgatgagcagaatcattacccgtctacccggattggatatttcatgtcctgaaacagactctttaagttaa

SEQ ID NO: 14 - DNA, 2334 bp, synthetic construct
  source 1..2334: mol_type = other DNA
atgactcaaaatatcaggccgttaccccaattcaaatatcatcccaagccactggaaacaggcgcatttgaacaggataaaaccgtagagtgcgattgctgtgaacaacagacgtcagtttattactcgggtcccttttattgcgttgatgaagttgaacatctctgtccgtggtgtattgcggacggttctgctgctgattgggtgttgtttgaagtataagttgacatatctgtactaaaagatggcataaattggaagtgtaaggtggcatagtctagtatttaaccaaatggttaaatggttgactcaccgggaattcgagataaattgcactgaaatctagaggtcgaaattcacctcgaaagcaagctgataaaccgatacaattaaaggctccttttggagccttttttttggagattttcaacgtgaaaaaattattattcgcaattccaagctaattcacctcgaaagcaagctgataaaccgatacaattaaaggctccttttggagccttttttttggagattttcaacgtgaaaaaattattattcgcaattccaagctctgcctcgcgcgtttcggtgatgacggtgaaaacctctgacacatgcagctcccggagacggtcagcttgtctgtaagcggatgccgggagcagacaagcccgtcagggcgcgtcagcgggtttggcgggtgtcggggcgcagccatgacccagtcacgtagcgatagcggagtgtatgggctcgatcccctcggatcgatccccatgtaatgaataaaagcagtaattaatacatctgtttcatttgaagcgcgaaagctaaagttttcgcatttatcggatccggctgctaatcgagttaattaaactagtgagctcggtacccggggatcctctagaggtcgaaattcacctcgaaagcaagctgataaaccgatacaattaaaggctccttttggagcctttttttttggagattttcaacgtgaaaaaattattattcgcaattccaagctaattcacctcgaaagcaagctgataaaccgatacaattaaaggctccttttggagcctttttttttggagattttcaacgtgaaaaaattattattcgcaattccaagctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatgggctcgatcccctcggatccaattggtcttaattttccttaattatttttaaagttagactgatttagacttggaaaagcttaatgattggagagctaaattgactaatagtattcagtcaagtttaattagttttaagcgatccatgcataactatttctgtacaatgctatattttcaccaattaataatttttaatcaagcctacattatgaaatatactatacccatttgaactcttctatttgtaccattgtcggtagcaaaaacttatggggttttgaacgtcacttaaattgtaagcatttgcgatggaggcgcgtttagagtcaaccttgattctgatatgctccgaatttttggtaagaatataagtgtgagagtagctaatgtggatacgcctgagttaagggaaaaatgtgaaaatgaaataactcgttatcatgcaaagtgactaaggttataatcttccgtttatggcacatagcagccaactaaacttgacagtatttttatgtggttggctttataaaaccagcatttggtaacattatgccaatttttacttcaatattatgccaacatacactacactaacggagctgtagcacaataagctcgtttgtacttatgccaacttatacttcaaacaacattgggcatagaaggtgttgaatttgagtatgatgaagaggacgaatttgccggtattaagaacacatatcctgatgaaatgctgaaagagttggttgaacgcacgccaggttatcatggatggcagcaggaattctggctcgcgcattgtggcgatttctgtgtttttatcggctatgtgggctggaatgatataaaagatcgcctcgatgaatttgccaaccttgaagaagattgtgagaatttcggtattagaaattctgatctagctaaatgcctgcaaaagggtggtcattgtcagggttatctcttccgctgtctccactgcggcaagctgagactgtggggtgatttttcgtag

SEQ ID NO: 15 - DNA, 47 bp, synthetic construct
  source 1..47: mol_type = other DNA
ggttgactcaccgggaattcgagataaattgcactgaaatctagagg

SEQ ID NO: 16 - DNA, 52 bp, synthetic construct
  source 1..52: mol_type = other DNA
ctcgattagcagccggatccgataaatgcgaaaactttagctttcgcgcttc

SEQ ID NO: 17 - DNA, 28 bp, synthetic construct
  source 1..28: mol_type = other DNA
ggatccggctgctaatcgagttaattaa

SEQ ID NO: 18 - DNA, 20 bp, synthetic construct
  source 1..20: mol_type = other DNA
gaattcccggtgagtcaacc

SEQ ID NO: 19 - DNA, 81 bp, synthetic construct
  source 1..81: mol_type = other DNA
ctgtctgccggaaaagatcgggtcacagaaacgtgaactgccgagtaggcagctgaagttggatccgaattcgagctcggg

SEQ ID NO: 20 - DNA, 25 bp, synthetic construct
  source 1..25: mol_type = other DNA
agaacttatcggtggtggtgtcacg

SEQ ID NO: 21 - DNA, 84 bp, synthetic construct
  source 1..84: mol_type = other DNA
gtttctgtgacccgatcttttccggcagacagatttccagctgcctactcggcagttcacccatggtatatctccttttaaagt

SEQ ID NO: 22 - DNA, 25 bp, synthetic construct
  source 1..25: mol_type = other DNA
cgtgacaccaccaccgataagttct

SEQ ID NO: 23 - DNA, 81 bp, synthetic construct
  source 1..81: mol_type = other DNA
aaagtttgcaggtagttttcaggatgatgccagtgaactgccgagtaggcagctgaagttggatccgaattcgagctcggg

SEQ ID NO: 24 - DNA, 25 bp, synthetic construct
  source 1..25: mol_type = other DNA
agaacttatcggtggtggtgtcacg

SEQ ID NO: 25 - DNA, 84 bp, synthetic construct
  source 1..84: mol_type = other DNA
tggcatcatcctgaaaactacctgcaaactttatttccagctgcctactcggcagttcacccatggtatatctccttttaaagt

SEQ ID NO: 26 - DNA, 25 bp, synthetic construct
  source 1..25: mol_type = other DNA
cgtgacaccaccaccgataagttct

SEQ ID NO: 27 - DNA, 22 bp, synthetic construct
  source 1..22: mol_type = other DNA
cggaagctttaatgcgggtcct

SEQ ID NO: 28 - DNA, 26 bp, synthetic construct
  source 1..26: mol_type = other DNA
gatgcgtccggcgtagaggatccacc

SEQ ID NO: 29 - DNA, 20 bp, synthetic construct
  source 1..20: mol_type = other DNA
ggcgagtttacgggttgtta

SEQ ID NO: 30 - DNA, 30 bp, synthetic construct
  source 1..30: mol_type = other DNA
gcgctcaaaggtaagtaaagggagtttgat

SEQ ID NO: 31 - DNA, 28 bp, synthetic construct
  source 1..28: mol_type = other DNA
tgagtagcccaaagataaagtatcccgc

SEQ ID NO: 32 - DNA, 27 bp, synthetic construct
  source 1..27: mol_type = other DNA
ctactcaaaagcagacaggatgtttct

SEQ ID NO: 33 - DNA, 25 bp, synthetic construct
  source 1..25: mol_type = other DNA
cggcctgttctcattatttaaataa

SEQ ID NO: 34 - DNA, 32 bp, synthetic construct
  source 1..32: mol_type = other DNA
tcatccaacagatgttgatttcacccatccac

SEQ ID NO: 35 - DNA, 32 bp, synthetic construct
  source 1..32: mol_type = other DNA
ttctcagctgtgttgattttcgtggtggtatg

SEQ ID NO: 36 - DNA, 32 bp, synthetic construct
  source 1..32: mol_type = other DNA
aaaagataagtatatcgtccgtgacgatacca

SEQ ID NO: 37 - DNA, 32 bp, synthetic construct
  source 1..32: mol_type = other DNA
tgaagaagtggttattgcgcgttgccccctgg